(19) United States
(12) Patent Application Publication
(10) Pub. No.: US 2015/0190391 A1
(43) Pub. Date: Jul. 9, 2015

(54) INHIBITORS OF NKX2.5 FOR VASCULAR REMODELLING

(71) Applicant: UCL Business PLC, London (GB)

(72) Inventors: David John Abraham, London (GB); Markella Ponticos, London (GB); Alan Matthew Holmes, London (GB)

(73) Assignee: UCL Business PLC, London (GB)

(21) Appl. No.: 14/415,558

(22) PCT Filed: Jul. 18, 2013
(86) PCT No.: PCT/GB2013/051916

§ 371 Date: Jan. 16, 2015

(30) Foreign Application Priority Data
Jul. 18, 2012 (GB) 1212773.4

(51) Int. Cl. A61K 31/4745; C12N 15/113

(52) U.S. Cl. CPC A61K 31/4745; C12N 15/113; C12N 2310/141; C12N 2310/14

(57) ABSTRACT

The invention relates to vascular remodelling, and to the treatment of conditions that are characterised or caused by inappropriate vascular remodelling. The invention also extends to pharmaceutical compositions for use in treating such conditions, and to methods of treatment.

The present invention relates to vascular remodelling, and particularly, although not exclusively, to the treatment of conditions that are characterised or caused by inappropriate vascular remodelling. The invention also extends to pharmaceutical compositions for use in treating such conditions, and to methods of treatment.

Vascular remodelling of resistance arteries is defined as the structural changes such as media thickening, reduced lumen diameter and consequent increased media:lumen ratio that can cause or occur as a result of vascular pathology. Several processes underlie these changes, including altered vascular smooth muscle cell (VSMC) growth, migration, differentiation and increased extracellular matrix abundance. Another factor contributing to vascular remodelling is inflammation, associated with macrophage infiltration, fibrosis/scarring, plaque formation and increased expression of redox-sensitive pro-inflammatory genes. Vascular remodelling is observed in vascular pathology associated with pulmonary hypertension and atherosclerosis, as well as other diseases, including coronary artery disease (CAD), peripheral arterial disease (PAD), chronic limb ischemia and stroke.

Pulmonary hypertension (PH) is an increase in the blood pressure of greater than 25 mmHg at rest or 30 mmHg with exercise in the pulmonary arteries, veins, or capillaries, collectively known as the lung vasculature. First indentified in 1891, PH is classified into a number of groups, including WHO Group I: Pulmonary arterial hypertension (PAH). Pulmonary hypertension (PH) is a hallmark of PAH, but PH includes all cases of increased pulmonary arterial pressure (PAP), regardless of its cause. The etiology and pathobiology of this rapidly progressive and ultimately fatal disease is complex and poorly understood with current treatments relatively ineffective. A disease of the small and medium pulmonary arteries, pulmonary arterial hypertension (PAH) is characterized by vasoconstriction and vascular proliferation and remodeling. All these processes are believed to contribute to the progressive increase in pulmonary vascular resistance in PAH, right ventricular hypertrophy and ultimately right heart failure. The intense remodeling of the pulmonary arteries is believed to result from matrix deposition and proliferation of resident cells, including pulmonary arterial smooth muscle (PASMCs), fibroblasts and angiogenic-endothelial cells.

PAH includes a heterogeneous group of conditions which, despite the diversity, is defined by similarities in pathophysiological, histological and prognostic features. A variety of conditions can lead to the development of PAH, such as congenital heart defects, chronic hypoxia, connective tissue diseases (systemic sclerosis, CREST syndrome, systemic lupus erythematosus, Sjögren's syndrome, rheumatoid arthritis, Takayasu's arteritis, polymyositis, dermatomyositis), and inflammation (acquired immunodeficiency syndrome and human immunodeficiency virus infection), ingestion of substances such as toxins. Finally, idiopathic PAH (IPAH) is a condition diagnosed when all other known causes of PAH have been ruled out.

Atherosclerosis is also major health concern, especially in the developed world, and an increasing burden to emerging economies. The atherosclerotic lesion develops progressively within the vessel wall in a chronic inflammatory and hyperlipidaemic environment. Endothelial cell damage promotes the recruitment of circulating monocytes which accumulate in the evolving lesion and eventually become foam cells within the intima. Progression of these primordial fatty streaks into mature plaques involves the extensive accumulation of inflammatory cells and the coalescence of lipid to form a lipid-rich core. Activated vascular smooth muscle cells enshroud the lipid core, forming a fibrous cap as part of the inherent vascular reparative process and vascular remodelling. Although composed of a complex mixture of extracellular matrix proteins, the collagens, notably type I, constitute a major component (˜60%) of total plaque protein. Collagen type I is predominantly produced by activated vascular smooth muscle cells, in the shoulder region or fibrous cap of the plaque is of primary importance in stabilising the plaque and protecting against plaque rupture. Collagen not only confers biomechanical strength and a structural framework, maintaining plaque integrity, but also influences macrophage activity, smooth muscle cell migration and proliferation, and when assembled into the extracellular matrix, functions as a reservoir for cytokines and growth factors. Mature collagen type I is subjected to degradation by matrix modifying enzymes, notably the matrix metalloproteinases.

In view of above, there is, therefore, clearly a need to provide alternative treatments for diseases which are characterised by vascular remodelling, such as atherosclerosis and Pulmonary Hypertension. To this end, the inventors investigated the role of the transcription factor, Nkx2.5, in such diseases. They demonstrated that Nkx2-5 is not expressed by normal vessels, but that, surprisingly, it is expressed in remodelling vessels and during vessel pathology. The inventors are therefore the first group to demonstrate that Nkx2-5 is essential in the pathological development of vascular remodelling diseases, and that inhibitors of this transcription factor will have important therapeutic implications in the treatment of diseases with vascular remodelling components.

Therefore, in a first aspect of the invention, there is provided an inhibitor of Nkx2.5 activity, for use in therapy or diagnosis.

In a second aspect, there is provided an inhibitor of Nkx2.5 activity, for use in the treatment, amelioration or prevention of a disease characterised by inappropriate vascular remodelling.

In a third aspect, there is provided a method of treating, ameliorating or preventing a disease characterised by inappropriate vascular remodelling in a subject, the method comprising administering, to a subject in need of such treatment, a therapeutically effective amount of an inhibitor of Nkx2.5 activity.

Nkx2-5, also known as Cardiac specific homeobox (CSX), belongs to the NK-2 family of homeobox DNA binding transcriptional activators that are structurally and functionally conserved in evolution. Nkx2-5 homologues have been described in many vertebrates from Xenopus to human and share a highly conserved protein structure of four distinct domains. The homeodomain has been well-characterized: it binds to a consensus DNA sequence through a helix-turn-helix motif and interacts with other transcription factors. The function(s) of the other domains is less well described, although it is known that Nkx2-5, apart from being a transcriptional activator, also has the ability to repress transcriptional activity through protein-protein interactions.

The genomic DNA sequence encoding human Nkx2-5 is located on Chromosome 5 (NC_{—}000005.9, NT 023133.13 (172659107 . . . 172662315, complement) and gives rise to three transcripts, namely the main transcript for Nkx2-5 (Isoform 1, Accession Number: NM_{—}004387) and two potential variant transcripts (Isoform 2, Accession Number: NM_{—}001166175.1; and Isoform 3, Accession Number: NM_{—}001166176.1), which are provided below.

The main transcript for Nkx2.5 (Accession Number: NM_{—}004387; gi|224589817:c172662315-172659107; Homo sapiens; chromosome 5, GRCh37.p5 Primary Assembly) is provided herein as SEQ ID No:1:

[SEQ ID NO: 1]
GCTCCTGTCATCGAGGCCCCTGGCCCAATGGCAGGCTGAGTCCCCCTCCT
CTGGCCTGGTCCCGCCTCTCCTGCCCCTTGTGCTCAGCGCTACCTGCTGC
CCGGACACATCCAGAGCTGGCCGACGGGTGCGCGGGCGGGCGGCGGCACC
ATGCAGGGAAGCTGCCAGGGGCCGTGGGCAGCGCCGCTTTCTGCCGCCCA
CCTGGCGCTGTGAGACTGGCGCTGCCACCATGTTCCCCAGCCCTGCTCTC
ACGCCCACGCCCTTCTCAGTCAAAGACATCCTAAACCTGGAACAGCAGCA
GCGCAGCCTGGCTGCCGCCGGAGAGCTCTCTGCCCGCCTGGAGGCGACCC
TGGCGCCCTCCTCCTGCATGCTGGCCGCCTTCAAGCCAGAGGCCTACGCT
GGGCCCGAGGCGGCTGCGCCGGGCCTCCCAGAGCTGCGCGCAGAGCTGGG
CCGCGCGCCTTCACCGGCCAAGTGTGCGTCTGCCTTTCCCGCCGCCCCCG
CCTTCTATCCACGTGCCTACAGCGACCCCGACCCAGCCAAGGACCCTAGA
GCCGAAAAGAAAGGTGAGGAGGAAACACAGGCCCCCTTCTCCCCTCCTGG
GTCGCTTTCGTCCCCAAGAAACTCAGGGCCAGGAGGAGGAGACACGCGCC
CTTGGGCCGAGGGCTGGGCTGCGGCGGGGGGTTCAGAATGTAAGATGCCT
GGTGTTGTCGCCAGGCTCCCGCGCCCCGCGTCCAATCGGAGGTTCAGAGG
AAATGCCGGATTGAAAGGATCAGAAGCAAGAGACCAAAAAACGTTTCCCC
CCGGCCTAACAAAGCCCCGGGCGGCTTCGGCTCTGCTCCTGGGTCTGGTA
GGAAGTTGAGAAATCGGTTTATGGTAGACAGAACAGAGAGACAAGCAGAT
AATCTCTGTTTTTAAATCTCCTTTGGATTTACGAATCTTTTTAAAGATCT
GATGAGAACCGCTAAACAGAAATTGAAATGTTGCTCACCAGACAGCTTTT
GCGTACAATCGGAGGAGGGTCCTGGACCTTCTTTCTGCAGCCCACCCACG
ACCCGGGTTTCTGGTGCCTTTCTTTCTTTGCGCCAGGAAAGTGGAGTCTG
GGATCGAGGGCCTTGATTTTAAAATGGGATACTGCGGACCCTCAGGAATC
TGACTTCACTTTATTTTTTCAGCACAACTTGCCGGCGCGGCCAGGGCGGA
GAGGTTCCCTCGTGGAAAAGTTAGGAAATGCTGCGCTACCGCGGGCACAA
GGGAGTGGACGAGATGAGTGCGGGATCATCCCGCAGGCCATCCCAGGATC
GGGGAGGGAGGCCGGCCCCGCTGCAGAAAGGGGCCTTCTGGGAGACCCCC
CAGCCCAAGGCAGGAGCCCGGGCGATTCCCGGGAGGCCGCAGGCGCTGGG
CGAAGCGCTGGGCGAAGGGCCGCTGCCAGCCGGGAGAGAATTCATAGGTT
TGTTGAGGAGCAGAGGCCTGGGAACAAATTCGGGCGGGCACGGCGGCTAG
AACTGATCGCTACCAATTCGAGGAAGCCAGCAAGGCAGGTTCCGAGGCCG
CCTGCCCACCCGCAGCTTCTTGGACACTGCGCAAACCCTGCTGCGGCCAG
GCTGGAGCCTCCGATCACCAAACCAACACTCCCTGGCCTTCTGTTTCTTG
ATTCCTTAATTTTGAGATAAGACCGTCCCTAGCAGTGAGGCCTCGGCCTC
TGTTCATTTAACTTCTCAAACCAAACTAGCCCTAATTCAGTTCACCCCAG
AGCATCACCTGGTTTTATTTTTATTTTTTTATTTTTTTATTTATTTTTTT
TTTTTTTGCAGCCTGAAATTTTAAGTCACCGTCTGTCTCCCTCACCAGGG
TGTGAACTGCCCCGAGGGCAGAGACCTCCCGTTTTGTTCTCCAGCGCCTT
GAGCCAGCCTGACTTTCTACAAATGCTGAGTGAGACGTGTCGGTGGCTCC
CAGTGCACTTGGCAGAGTGAGCCGCAGCCAGCTGGGCGCTCCAGGCAGGA
CACAGTGGCCTCCACGAGGATCCCTTACCATTACTGTGCGGCCGCGCTCC
GTAGGTCAAGCCGCTCTTACCAAGCGTCTCTCTGCCTCTCTGTTCCCCCT
CAGAGCTGTGCGCGCTGCAGAAGGCGGTGGAGCTGGAGAAGACAGAGGCG
GACAACGCGGAGCGGCCCCGGGCGCGACGGCGGAGGAAGCCGCGCGTGCT
CTTCTCGCAGGCGCAGGTCTATGAGCTGGAGCGGCGCTTCAAGCAGCAGC
GGTACCTGTCGGCCCCCGAACGCGACCAGCTGGCCAGCGTGCTGAAACTC
ACGTCCACGCAGGTCAAGATCTGGTTCCAGAACCGGCGCTACAAGTGCAA
GCGGCAGCGGCAGGACCAGACTCTGGAGCTGGTGGGGCTGCCCCCGCCGC
CGCCGCCGCCTGCCCGCAGGATCGCGGTGCCAGTGCTGGTGCGCGATGGC
AAGCCATGCCTAGGGGACTCGGCGCCCTACGCGCCTGCCTACGGCGTGGG
CCTCAATCCCTACGGTTATAACGCCTACCCCGCCTATCCGGGTTACGGCG
GCGCGGCCTGCAGCCCTGGCTACAGCTGCACTGCCGCTTACCCCGCCGGG
CCTTCCCCAGCGCAGCCGGCCACTGCCGCCGCCAACAACAACTTCGTGAA
CTTCGGCGTCGGGGACTTGAATGCGGTTCAGAGCCCCGGGATTCCGCAGA
GCAACTCGGGAGTGTCCACGCTGCATGGTATCCGAGCCTGGTAGGGAAGG
GACCCGCGTGGCGCGACCCTGACCGATCCCACCTCAACAGCTCCCTGACT
CTCGGGGGGAGAAGGGGCTCCCAACATGACCCTGAGTCCCCTGGATTTTG
CATTCACTCCTGCGGAGACCTAGGAACTTTTTCTGTCCCACGCGCGTTTG
TTCTTGCGCACGGGAGAGTTTGTGGCGGCGATTATGCAGCGTGCAATGAG
TGATCCTGCAGCCTGGTGTCTTAGCTGTCCCCCCAGGAGTGCCCTCCGAG
AGTCCATGGGCACCCCCGGTTGGAACTGGGACTGAGCTCGGGCACGCAGG
GCCTGAGATCTGGCCGCCCATTCCGCGAGCCAGGGCCGGGCGCCCGGGCC
TTTGCTATCTCGCCGTCGCCCGCCCACGCACCCACCCGTATTTATGTTTT
TACCTATTGCTGTAAGAAATGACGATCCCCTTCCCATTAAAGAGAGTGCG
TTGACCCCG

The protein sequence of human Nkx2-5 isoform 1 (Accession Number: NP_{—}004378.1) is provided herein as SEQ ID No:2, as follows:

[SEQ ID NO: 2]
MFPSPALTPTPFSVKDILNLEQQQRSLAAAGELSARLEATLAPSSCMLAA
FKPEAYAGPEAAAPGLPELRAELGRAPSPAKCASAFPAAPAFYPRAYSDP
DPAKDPRAEKKELCALQKAVELEKTEADNAERPRARRRRKPRVLFSQAQV
YELERRFKQQRYLSAPERDQLASVLKLTSTQVKIWFQNRRYKCKRQRQDQ
TLELVGLPPPPPPPARRIAVPVLVRDGKPCLGDSAPYAPAYGVGLNPYGY
NAYPAYPGYGGAACSPGYSCTAAYPAGPSPAQPATAAANNNFVNFGVGDL
NAVQSPGIPQSNSGVSTLHGIRAW

The protein sequence of human Nkx2-5 isoform 2 (Accession Number: NP_{—}001159647.1; >gi|260898750|ref|NP_{—}001159647.1| homeobox protein Nkx-2.5 isoform 2; Homo sapiens), is provided herein as SEQ ID No:3, as follows.

[SEQ ID NO: 3]
MFPSPALTPTPFSVKDILNLEQQQRSLAAAGELSARLEATLAPSSCMLAA
FKPEAYAGPEAAAPGLPELRAELGRAPSPAKCASAFPAAPAFYPRAYSDP
DPAKDPRAEKKA

The protein sequence of human Nkx2-5 isoform 3 (Accession Number: NP_{—}001159648.1; >gi|2608987521ref|NP_{—}001159648.1| homeobox protein Nkx-2.5 isoform 2 (Homo sapiens, is provided herein as SEQ ID No:4, as follows.

[SEQ ID NO: 4]
MFPSPALTPTPFSVKDILNLEQQQRSLAAAGELSARLEATLAPSSCMLAA
FKPEAYAGPEAAAPGLPELRAELGRAPSPAKCASAFPAAPAFYPRAYSDP
DPAKDPRAEKKGCELPRGQRPPVLFSSALSQPDFLQMLSETCRWLPVHLA
E

Reference herein to Nkx2-5 herein refers to the protein identified as any one of SEQ ID No: 2, 3 or 4 (i.e. isoform 1, 2 or 3), and to functional variants and fragments thereof. However, isoform 1 of Nkx2-5 (SWISS-PROT. Acc. No. NP_{—}004378), and functional variants and fragments thereof, is preferred. Thus, preferably the inhibitor prevents or reduces expression of Nkx2-5, wherein Nkx2-5 comprises an amino acid sequence substantially as set out in any one of SEQ ID No: 2, 3 or 4, or a functional variant or fragment thereof.

As described in the Examples, the inventors have shown that Nkx2-5 is not expressed by normal vessels, but is expressed in remodelling vessels and during vessel pathology. Nkx2-5 has been observed in the human atherosclerotic lesions in the aorta, the coronary, the carotid and the femoral arteries, and it is also expressed in the pulmonary vasculature of patients with pulmonary arterial hypertension. Therefore, inhibitors of this transcription factor will be useful for treating diseases with vascular remodelling components.

For example, diseases characterised by inappropriate vascular remodelling (and deposition of vascular extracellular matrix), which may be treated, include pulmonary hypertension (PH), pulmonary arterial hypertension (PAH) including all types of PAH associated with connective tissue diseases or HW, atherosclerosis, coronary artery disease (CAD), peripheral arterial disease (PAD), chronic limb ischemia or stroke, renal artery disease, metabolic syndrome and diabetes, rheumatological diseases (e.g. systemic lupus erethematosus, systemic sclerosis, rheumatoid arthritis, vasculis), fibromuscular displasia, and aneurisms.

Inhibitors capable of decreasing the biological activity of Nkx2-5 may achieve their effect by a number of means. For instance, such inhibitors may:—
(a) reduce interaction between Nkx2-5 and nucleic acid and/or other transcription factors;
(b) compete with endogenous Nkx2-5 for nucleic acid binding and/or other transcription factor binding;
(c) bind to Nkx2-5 to reduce its biological activity;
(d) decrease the expression of Nkx2-5; or
(e) inhibit Nkx2-5 translocation to the nucleus.

In one embodiment, the inhibitor may directly interact with Nkx2-5, e.g. (a) to (c) above. Preferred inhibitors for use according to the invention may comprise small molecule inhibitors, which may be identified as part of a high throughput screen of small molecule libraries. For example, the inhibitor may comprise an antibody raised against Nkx2-5, i.e. an Nkx2-5 neutralising antibody. The antibody may be polyclonal or monoclonal. Conventional hybridoma techniques may be used to raise the antibodies, and are well-known in the art. The homeodomain of Nkx2-5 is essential as it binds DNA. The C-terminal binds and interacts with other transcription factors, but also contains inhibitory activity. Thus, the antigen used to generate monoclonal antibodies according to the present invention may be the whole Nkx2-5 protein or a fragment thereof.

In another embodiment, the inhibitor according to the invention may comprise an inactive peptide fragment of Nkx2-5, which competes with endogenous Nkx2-5 and thereby reduces its activity. For instance, truncation mutants of Nkx2-5 that do not bind to nucleic acid or other transcription factors, and which inhibit the ability of Nkx2-5 to bind nucleic acid, may also be used as inhibitors of the invention.

In another embodiment, the inhibitor may prevent or reduce expression of Nkx2-5 (i.e. (d) above).

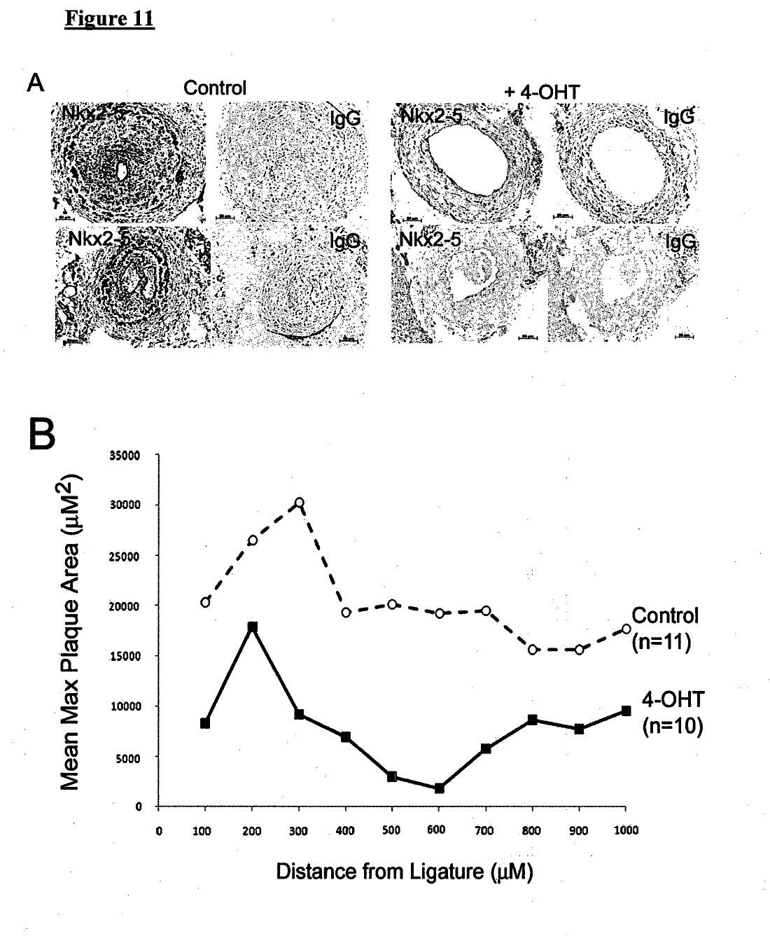

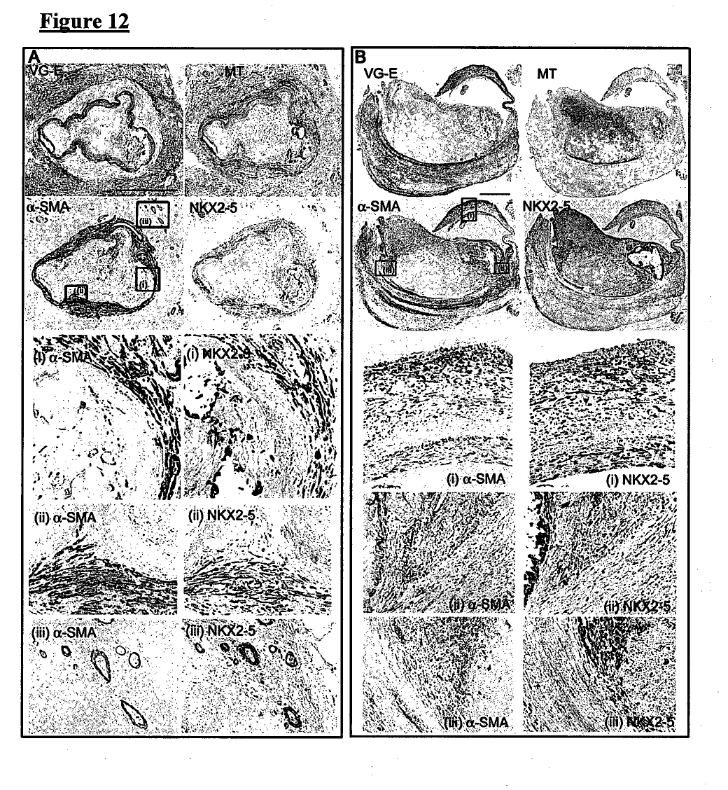

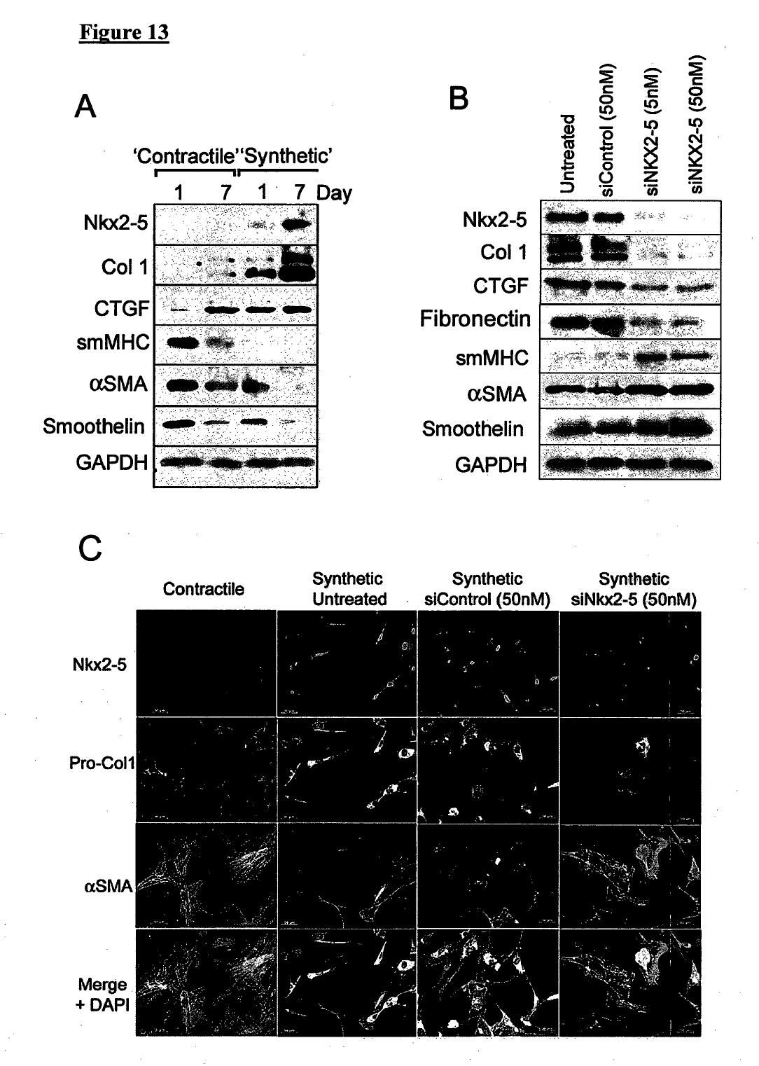

As described in the Examples, the inventors have demonstrated that inhibition of Nkx2-5 expression by siRNA in ‘synthetic’ aortic smooth muscle cells not only down-regulates collagen type I protein levels, but also other proteins associated with the extracellular matrix that are known to be upregulated as part of the fibrogenic response (CTGF, Fibronectin). The inventors have also shown that Nkx2-5 knockdown results in the up-regulation of the expression of proteins associated with the normal differentiated, contractile state of the smooth muscle cell (e.g. α-SMA, sm-MHC, smoothelin). As shown in FIG. 13C, four colour immunofluorescent staining of Human aortic smooth muscle cells reveals that inhibition of Nkx2-5 using siRNA results in down-regulation of procollagen type I and up-regulation of αSMA fibres and a visible change in smooth muscle cell phenotype. These data suggest that Nkx2-5 is directly involved in the de-differentiation of vascular smooth muscle cells from the differentiated contractile phenotype to the de-differentiated synthetic phenotype associated with vascular pathology, remodelling and scarring.

Therefore, the inhibitor according to the invention may be a gene-silencing molecule.

The term “gene-silencing molecule” can mean any molecule that interferes with the expression of the Nkx2-5 gene. Such molecules include, but are not limited to, RNAi molecules, including siNA, siRNA, miRNA, ribozymes and antisense molecules. The use of such molecules represents an important aspect of the invention.

Therefore, according to a fourth aspect of the present invention, there is provided an Nkx2-5 gene-silencing molecule for use in the treatment, amelioration or prevention of a disease characterised by inappropriate vascular remodelling.

Gene-silencing molecules may be antisense molecules (antisense DNA or antisense RNA) or ribozyme molecules. Ribozymes and antisense molecules may be used to inhibit the transcription of the Nkx2-5 gene. Antisense molecules are oligonucleotides that bind in a sequence-specific manner to nucleic acids, such as DNA or RNA. When bound to mRNA that has a complimentary sequence, antisense RNA prevents translation of the mRNA. Triplex molecules refer to single antisense DNA strands that bind duplex DNA forming a colinear triplex molecule, thereby preventing transcription. Particularly useful antisense nucleotides and triplex molecules are ones that are complimentary to, or bind, the sense strand of DNA (or mRNA) that encodes Nkx2-5.

The expression of ribozymes, which are enzymatic RNA molecules capable of catalysing the specific cleavage of RNA substrates, may also be used to block protein translation. The mechanism of ribozyme action involves sequence specific hybridisation of the ribozyme molecule to complementary target RNA, followed by endonucleolytic cleavage, e.g. hammerhead motif ribozymes.

It is preferred that the gene-silencing molecule is a short interfering nucleic acid (siNA). The siNA molecule may be double-stranded and therefore comprises a sense and an antisense strand. The siNA molecule may comprise an siDNA molecule or an siRNA molecule. However, it is preferred that the siNA molecule comprises an siRNA molecule. Hence, the siNA molecule according to the invention preferably down-regulates gene expression by RNA interference (RNAi).

RNAi is the process of sequence specific post-transcriptional gene-silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted ‘knockdown’ of gene expression.

Preferably, the siNA molecule is substantially identical with at least a region of the coding sequence of the Nkx2-5 gene (see above) to enable down-regulation of the gene. Preferably, the degree of identity between the sequence of the siNA molecule and the targeted region of the Nkx2-5 gene is at least 60% sequence identity, preferably at least 75% sequence identity, preferably at least 85% identity, preferably at least 90% identity, preferably at least 95% identity, preferably at least 97% identity, and most preferably at least 99% identity.

The siNA molecule may comprise between approximately 5 bp and 50 bp, more preferably between 10 bp and 35 bp, even more preferably between 15 bp and 30 bp, and yet still more preferably, between 16 bp and 25 bp. Most preferably, the siNA molecule comprises less than 22 bp.

Design of a suitable siNA molecule is a complicated process, and involves very carefully analysing the sequence of the target mRNA molecule. Using considerable inventive endeavour, the inventors have chosen a defined sequence of siRNA which has a certain composition of nucleotide bases, which they have shown has the required affinity and also stability to cause the RNA interference. The siNA molecule may be either synthesised de novo, or produced by a micro-organism. For example, the siNA molecule may be produced by bacteria, for example E. coli.

It will be appreciated that such siNAs may comprise uracil (siRNA) or thymine (siDNA). Accordingly, the nucleotides U and T, as referred to above, may be interchanged. However, it is preferred that siRNA is used.

Especially preferred siNA molecule sequences, which are adapted to down-regulate expression of the gene encoding Nkx2-5 may comprise the following sequences:—

(SEQ ID No. 5)
5′ - CCTCAATCCCTACGGTTAT - 3′;
(SEQ ID No. 6)
5′ - CCAACAACAACTTCGTGAA - 3′;
(SEQ ID No. 7)
5′ - GCTACAAGTGCAAGCGGCA - 3′;
and
(SEQ ID No. 8)
5′ - CCGGGATTCCGCAGAGCAA - 3′.

The siRNA of any of SEQ ID No. 5-8 can be used as a siNA molecule for use according to the present invention.

The inventors tested each of these siNA molecules by the methods as described in the Examples, and demonstrated that these inhibitors were effective for reducing Nkx2-5 expression, and are thereby effective for treating diseases characterized by inappropriate vascular remodelling.

The inventors also believe that microRNAs (miRNA) may be used as a suitable siNA molecule for use according to the invention. The miRNA may be selected from: miR-125b, miR-145, miR-143, miR-367, miR-384, miR-363, miR-32, miR-25, and miR-92a and b.

Gene-silencing molecules used according to the invention are preferably nucleic acids (e.g. siRNA, miRNA, antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene-silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage or virus DNA. In addition to, or instead of using the vector to synthesise the gene-silencing molecule, the vector may be used as a delivery system for transforming a target cell with the gene-silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, vasculature cells. The promoter may be constitutive or inducible.

Alternatively, the gene-silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentovirus and the like). Alternatively a “naked” siNA or antisense molecule may be inserted into a subject's cells by a suitable means e.g. direct endocytotic uptake.

The gene-silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing an siNA molecule; viral vectors comprising a gene-silencing sequence or means of providing direct nucleic acid uptake (e.g. endocytosis) by application of the gene-silencing molecule directly.

In a preferred embodiment of the present invention, siNA molecules may be delivered to a target cell (whether in a vector or “naked”) and may then rely upon the host cell to be replicated and thereby reach therapeutically effective levels. When this is the case, the siNA is preferably incorporated in an expression cassette that will enable the siNA to be transcribed in the cell and then interfere with translation (by inducing destruction of the endogenous mRNA coding Nxk2-5).

In another embodiment, the inhibitor may inhibit of Nkx2-5 translocation to the nucleus (i.e. (e) above).

As described in the Examples, the inventors have also shown that Nkx2-5 is regulated by phosphorylation by casein kinase II (CK II), and that NKX2-5 phosphorylation results in a surprising decrease of NKX2-5 nuclear translocation and DNA binding ability.

Accordingly, the inhibitor according to the invention may be capable of inhibiting casein kinase II (CK II) activity.

Examples of preferred CK II inhibitors which may be used as an inhibitor of the invention include: CX-4945 [CAS number 1009820-21-6], CX-8184, Casein Kinase II Inhibitor III, (TBCA) [CAS 934358-00-6], CKII inhibitor IV (IQA) [CAS 391670-48-7] Casein Kinase II Inhibitor V, (Quinalizarin) [CAS 81-61-8], Casein Kinase II Inhibitor VI, (TMCB) [CAS 905105-89-7], Casein Kinase II Inhibitor VII, and Casein Kinase II Inhibitor VIII.

As described in the Examples, CX-4945 has been shown to inhibit Nkx2-5 nuclear translocation at a concentration range 1-10 nM in vascular smooth muscle cells. It blocks phenotypic modulation from a normal ‘contractile’ to the ‘synthetic’ vascular smooth muscle cells phenotype associated with vascular remodelling and pathology and inhibits vascular smooth muscle cell migration.

Thus, a most preferred CK II inhibitor comprises CX-4945.

It will be appreciated that inhibitors according to the invention may be used in a medicament, which may be used in a monotherapy, i.e. use of only an inhibitor (e.g. antibody, siNA molecule or CX-4945) for treating, ameliorating, or preventing a disease condition characterised by inappropriate vascular remodelling. Alternatively, inhibitors according to the invention may be used as an adjunct to, or in combination with, known therapies for treating, ameliorating, or preventing diseases characterised by inappropriate vascular remodelling. For example, inhibitors of the invention may be used in combination with known agents for treating PH, such as diuretics, beta blockers, ACE inhibitors etc.

The inhibitors according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising inhibitors according to the invention may be used in a number of ways. For instance, oral administration may be required, in which case the inhibitors may be contained within a composition that may, for example, be ingested orally in the form of a tablet, capsule or liquid. Compositions comprising inhibitors of the invention may be administered by inhalation (e.g. intranasally). Compositions may also be formulated for topical use. For instance, creams or ointments may be applied to the skin, for example, adjacent the treatment site, e.g. the artery or vein displaying PH, PAH or atherosclerosis etc.

Inhibitors according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with inhibitors used according to the invention is required and which would normally require frequent administration (e.g. at least daily injection).

In a preferred embodiment, inhibitors and compositions according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the inhibitor that is required is determined by its biological activity and bioavailability, which in turn depends on the mode of administration, the physiochemical properties of the inhibitor and whether it is being used as a monotherapy or in a combined therapy. The frequency of administration will also be influenced by the half-life of the inhibitor within the subject being treated. Optimal dosages to be administered may be determined by those skilled in the art, and will vary with the particular inhibitor in use, the strength of the pharmaceutical composition, the mode of administration, and the advancement of the disease being treated. Additional factors depending on the particular subject being treated will result in a need to adjust dosages, including subject age, weight, gender, diet, and time of administration.

Generally, a daily dose of between 0.01 μg/kg of body weight and 0.5 g/kg of body weight of the inhibitor according to the invention may be used for treating, ameliorating, or preventing the disease characterised by inappropriate vascular remodelling, depending upon which inhibitor is used, e.g siNA, antibody or small molecule inhibitor such as CX-4945. More preferably, the daily dose of inhibitor is between 0.01 mg/kg of body weight and 500 mg/kg of body weight, more preferably between 0.1 mg/kg and 200 mg/kg body weight, and most preferably between approximately 1 mg/kg and 100 mg/kg body weight.

When the inhibitor is an siNA molecule, the daily dose may be between 1 μg/kg of body weight and 100 mg/kg of body weight, and more preferably, between approximately ion/kg and 10 mg/kg, and even more preferably, between about 50 μg/kg and 1 mg/kg. When the inhibitor (e.g. antibody or siNA) is delivered to a cell, daily doses may be given as a single administration (e.g. a single daily injection). Typically, a therapeutically effective dosage should provide about 1 ng to 100 μg/kg of the inhibitor per single dose, and preferably, 2 ng to song per dose. Antibody inhibitors may be administered in amounts between 10 μg/kg and 100 mg/kg, preferably in amounts between 100 μg/kg and 10 mg/kg, and more preferably may be administered at about 1 mg/kg. Such doses are particularly suitable when administered every few (e.g. every three) days.

The inhibitor may be administered before, during or after onset of the disease characterised by inappropriate vascular remodelling. Daily doses may be given as a single administration (e.g. a single daily injection). Alternatively, the inhibitor may require administration twice or more times during a day. As an example, inhibitors may be administered as two (or more depending upon the severity of the disease being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of inhibitors according to the invention to a patient without the need to administer repeated doses.

When the inhibitor is a nucleic acid, conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to form specific formulations comprising the inhibitor according to the invention and precise therapeutic regimes (such as daily doses of the inhibitor and the frequency of administration). The inventors believe that they are the first to describe a pharmaceutical composition for treating diseases characterised by inappropriate vascular remodelling, based on the use of the inhibitor of the invention.

Hence, in a fifth aspect of the invention, there is provided an inappropriate vascular remodelling treatment composition, comprising an inhibitor of Nkx2.5 activity and a pharmaceutically acceptable vehicle.

The term “inappropriate vascular remodelling treatment composition” can mean a pharmaceutical formulation used in the therapeutic amelioration, prevention or treatment of any disease condition characterised by inappropriate (i.e. too much or too little) vascular remodelling in a subject.

The invention also provides in a sixth aspect, a process for making the composition according to the fifth aspect, the process comprising contacting a therapeutically effective amount of an inhibitor of Nkx2.5 activity and a pharmaceutically acceptable vehicle.

The inhibitor may a small molecule inhibitor, an antibody, a gene-silencing molecule (e.g siNA) or a CKII inhibitor, such as CX-4945.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the inhibitor is any amount which, when administered to a subject, is the amount of medicament or drug that is needed to treat the condition characterised by inappropriate vascular remodelling, or produce the desired effect.

For example, the therapeutically effective amount of inhibitor used may be from about 0.01 mg to about 800 mg, and preferably from about 0.01 mg to about 500 mg. It is preferred that the amount of inhibitor is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the inhibitor of Nkx2.5 activity) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The inhibitor according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The inhibitor may be prepared as a sterile solid composition that may be dissolved or suspended at the time of administration using sterile water, saline, or other appropriate sterile injectable medium.

The inhibitors and pharmaceutical compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The inhibitors according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Knowledge of the surprising role that Nkx2-5 plays in inappropriate vascular remodelling diseases, such as PH, PAH, atherosclerosis, CAD, PAD, chronic limb ischemia and stroke etc. has enabled the inventors to develop a novel screening assay for identifying whether or not test compounds can act as useful inhibitors for treating or preventing any of these diseases, for example for inclusion in the pharmaceutical compositions described herein.

Thus, according to a seventh aspect, there is provided an assay for screening a test compound to test whether or not the compound has efficacy for treating or preventing a disease characterised by inappropriate vascular remodelling, the assay comprising:
(i) exposing a biological system to a test compound;
(ii) detecting the activity or expression of Nkx2-5 in the biological system; and
(iii) comparing the activity or expression of Nkx2-5 in the biological system treated with the test compound relative to the activity or expression of Nkx2-5 found in a control biological system that was not treated with the test compound,
   wherein a decreased level of activity or expression of Nkx2-5 in the presence of the test compound relative to that detected in the control biological system is an indication of the ability of the test compound to treat or prevent a disease characterised by inappropriate vascular remodelling.

It will be appreciated that the assay according to the seventh aspect may be adapted such that it is used to test whether or not a test compound actually causes a disease characterised by inappropriate vascular remodelling.

Therefore, according to an eighth aspect of the invention, there is provided an assay for screening a test compound to test whether or not the compound causes a disease characterised by inappropriate vascular remodelling, the assay comprising:
(i) exposing a biological system to a test compound;
(ii) detecting the activity or expression of Nkx2-5 in the biological system; and
(iii) comparing the activity or expression of Nkx2-5 in the biological system treated with the test compound relative to the activity or expression of Nkx2-5 found in a control biological system that was not treated with the test compound,
   wherein an increased level of activity or expression of Nkx2-5 in the presence of the test compound relative to that detected in the control biological system is an indication that the test compound causes a disease characterised by inappropriate vascular remodelling.

The assays of the invention are based upon the inventors' realisation that the extent of Nkx2-5 expression and/or activity may be closely related to the development of a disease characterised by inappropriate vascular remodelling. The screening assay of the seventh aspect is particularly useful for screening libraries of compounds to identify compounds that may be used as inhibitor used in the invention. The assay of the eighth aspect may be used to identify compounds that cause disease. Accordingly, the screen according to the eighth aspect of the invention may be used for environmental monitoring (e.g. to test effluents from factories) or in toxicity testing (e.g. to test the safety of putative pharmaceuticals, cosmetics, foodstuffs and the like).

The term “biological system” can mean any experimental system that would be understood by a skilled person to provide insight as to the effects a test compound may have on Nkx2-5 activity or expression in the physiological environment. The system may comprise: (a) an experimental test subject when an in vivo test is to be employed; (b) a biological sample derived from a test subject (for instance: blood or a blood fraction (e.g. serum or plasma), lymph or a cell/biopsy sample); (c) a cell line model (e.g. a cell naturally expressing Nkx2-5 or a cell engineered to express Nkx2-5); or (d) an in vitro system that contains Nkx2-5 or its gene and simulates the physiological environment such that Nkx2-5 activity or expression can be measured.

The screen preferably assays biological cells or lysates thereof. When the screen involves the assay of cells, they may be contained within an experimental animal (e.g. a mouse or rat) when the method is an in vivo based test. Alternatively, the cells may be in a tissue sample (for ex vivo based tests) or the cells may be grown in culture. It will be appreciated that such cells should express, or may be induced to express, functional Nkx2-5. It is also possible to use cells that are not naturally predisposed to express Nkx2-5 provided that such cells are transformed with an expression vector. Such cells represent preferred test cells for use according to the seventh and eighth aspects of the invention. This is because animal cells or even prokaryotic cells may be transformed to express human Nkx2-5 and therefore represent a good cell model for testing the efficacy of candidate drugs for use in human therapy.

It is most preferred that biological cells used according to the screening assays are derived from a subject displaying one example of a disease characterised by inappropriate vascular remodelling, such as PH or PAH or atherosclerosis.

With regards to “detecting the activity or expression of Nkx2-5” according to the screening assays described herein, the term “activity” can mean the detection of binding between Nkx2-5 and nucleic acid and/or other transcription factors; translocation or determination of an end-point physiological effect.

The term “expression” can mean the detection of the Nkx2-5 protein in any compartment of the cell (e.g. in the nucleus, cytosol, the Endoplasmic Reticulum or the Golgi apparatus); or detection of the mRNA encoding Nkx2-5.

Expression of Nkx2-5 in the biological system may be detected by Western blot, immuo-precipitation or immunohistochemistry. The screening assays may also be based upon the use of cell extracts comprising Nkx2-5. Such extracts are preferably derived from the cells described above.

The activity or expression of Nkx2-5 may be measured using a number of conventional techniques known to the skilled person. The test may be an immunoassay-based test. For instance, labelled antibodies may be used in an immunoassay to evaluate binding of a compound to Nkx2-5 in the sample. Nkx2-5 may be isolated and the amount of label bound to it detected. A reduction in bound label (relative to controls) would suggest that the test compound competes with the label for binding to Nkx2-5 and that it was also a putative therapeutic compound for use in treating disease.

Alternatively, a functional activity measuring Nkx2-5 activity may be employed. Furthermore molecular biology techniques may be used to detect Nkx2-5 in the screen. For instance, cDNA may be generated from mRNA extracted from tested cells or subjects and primers designed to amplify test sequences used in a quantitative Polymerase Chain Reaction to amplify from cDNA.

When a subject is used (e.g. an animal model or even an animal model engineered to express human Nkx2-5), the test compound should be administered to the subject for a predetermined length of time and then a sample taken from the subject for assaying Nkx2-5 activity or expression. The sample may for instance be blood or biopsy tissue.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the nucleotide sequence identified as SEQ ID No:1 (i.e. the DNA sequence encoding human Nkx2-5) or the protein identified as SEQ ID No:2 (i.e. human Nkx2-5 protein isoform 1), or 40% identity with the nucleotide identified as SEQ ID No:5-8 (i.e. an siRNA molecule for use in gene-silencing of Nkx2-5), and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No's: 1 or 4-8 or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequence shown in SEQ ID No:2-4.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

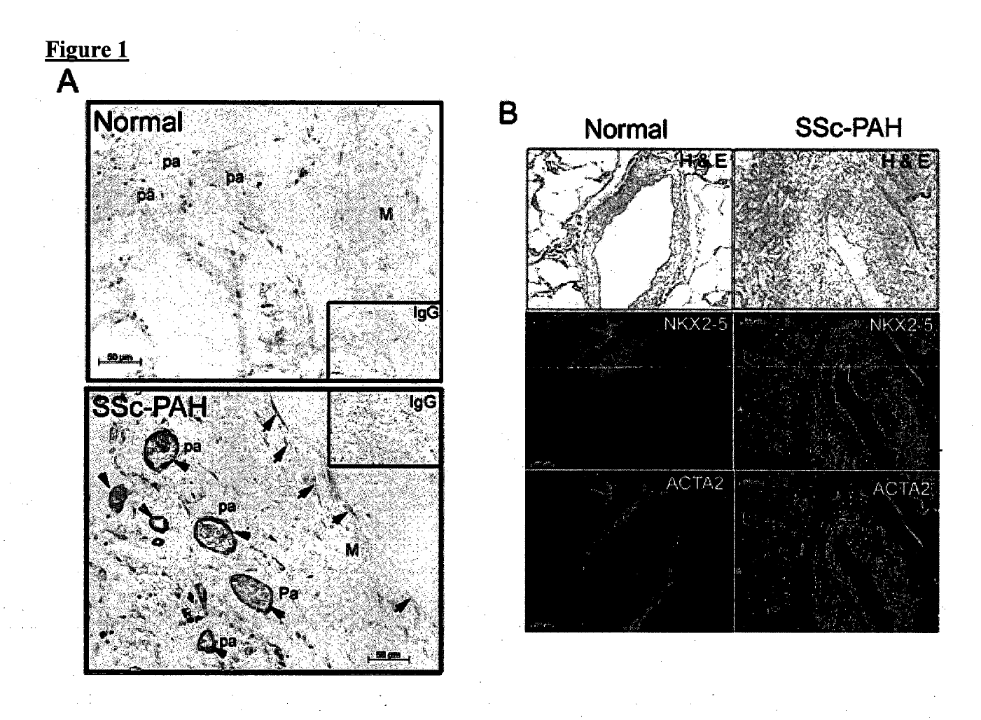

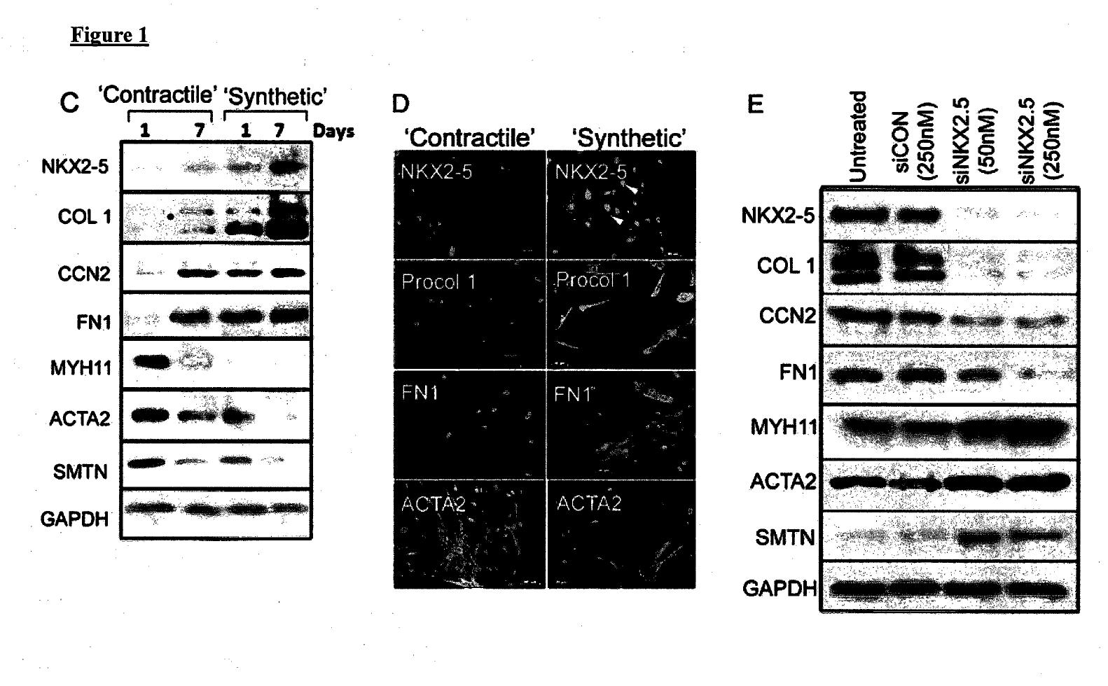

FIG. 1 shows that vascular expression of Nkx2-5 in human cells and tissues. Nkx2-5 is expressed in the pulmonary vasculature of SSc-PAH patients but not healthy controls (A). Nkx2-5 expression is observed in vascular smooth muscle cells (B) that are undergoing phenotypic modulation to a more ‘synthetic phenotype’ (C, D) which coincides with the overexpression of extracellular matrix proteins (COL1, CCN2, FN1) and the downrgulation of contractile proteins (ACTA2, MYH11) (C,D). Inhibition of Nkx2-5 expression in vitro using siRNA results in a decrease in ECM proteins and an increase in contractile protein expression (E);

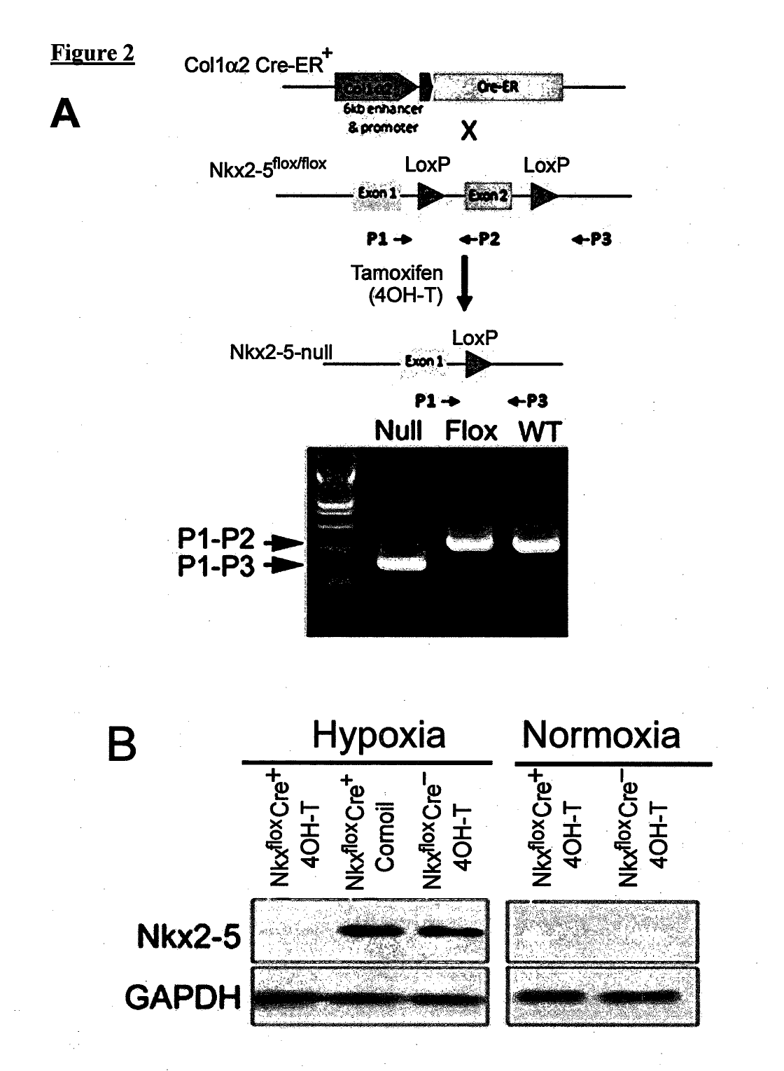

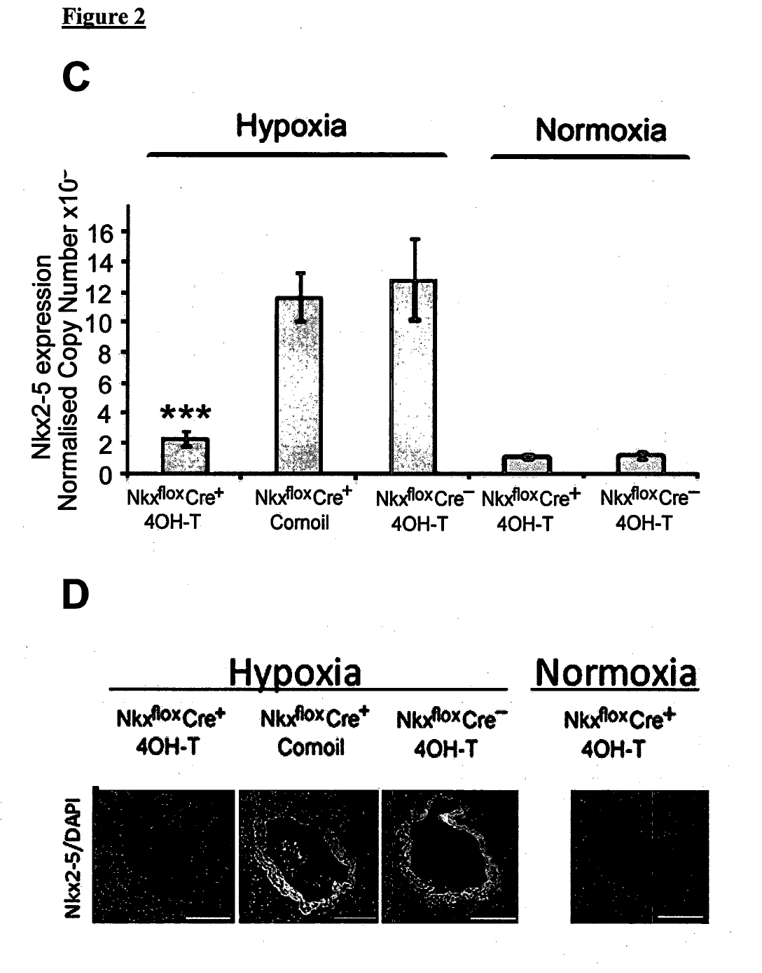

FIG. 2 shows the conditional inducible Nkx2-5 knockout mouse model in chronic hypoxia;

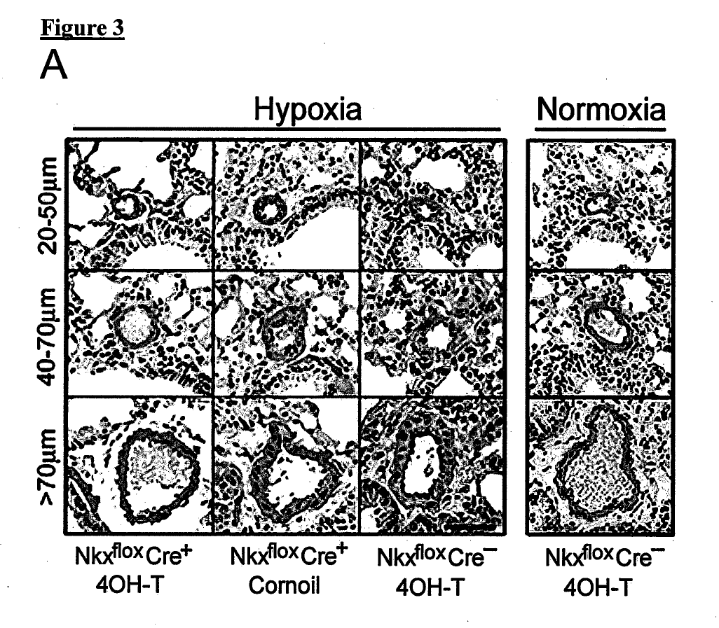

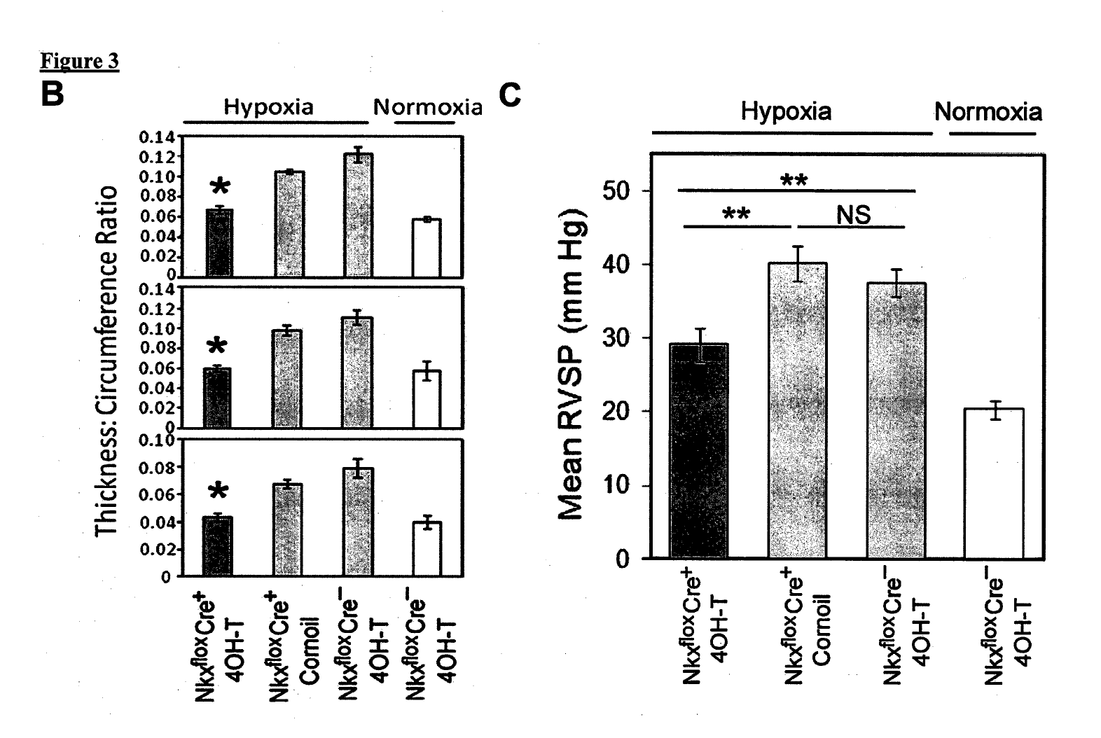

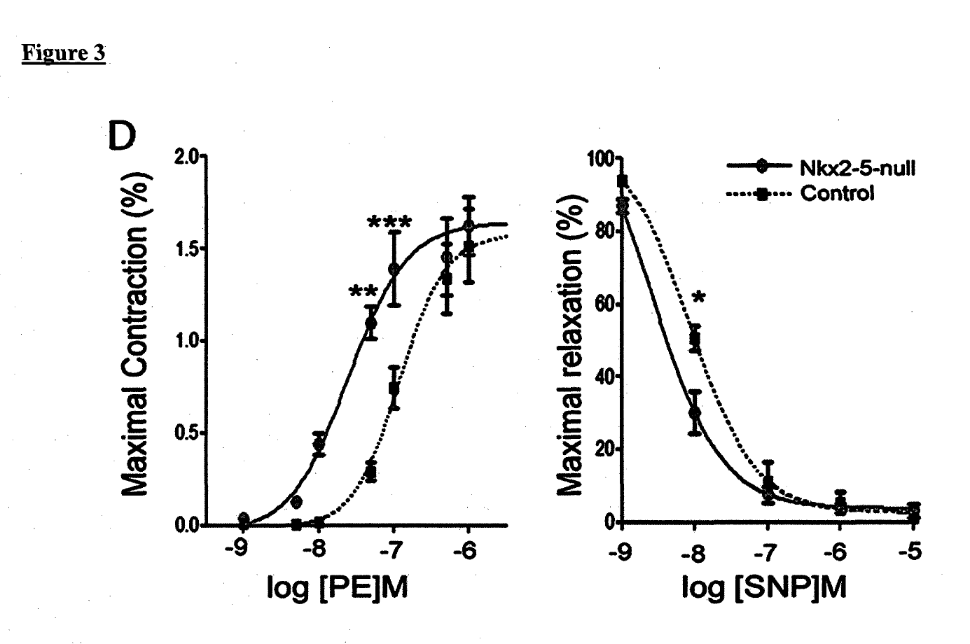

FIG. 3 shows that a lack of Nkx2-5 attenuates pulmonary vascular remodelling in the chronic hypoxia model of PAH;

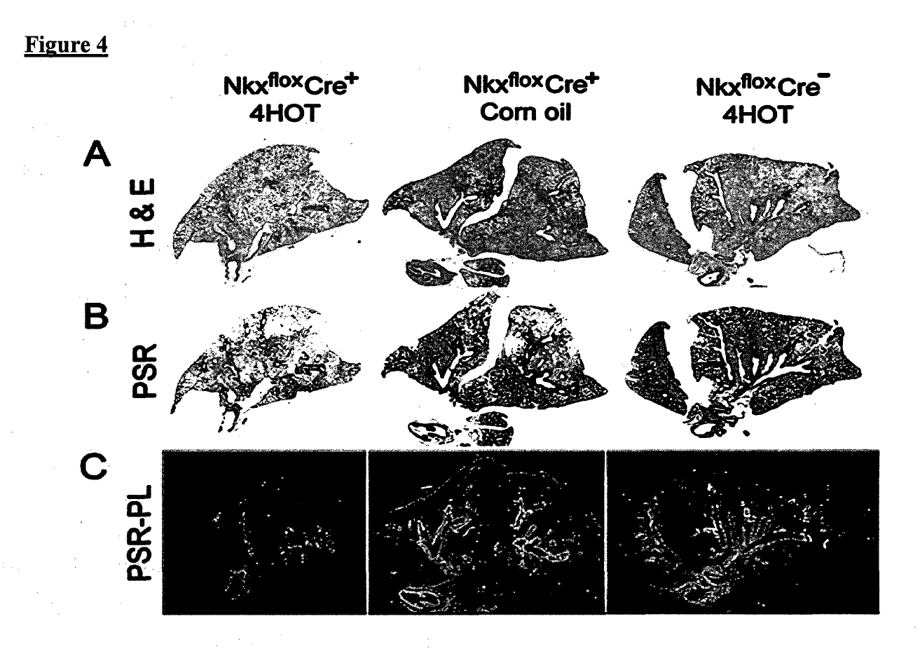

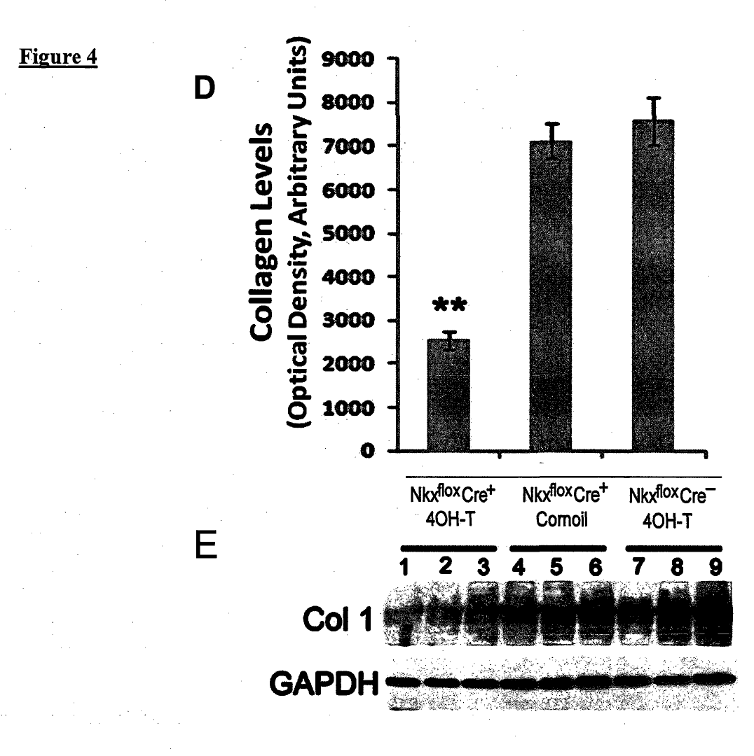

FIG. 4 shows that pulmonary collagen levels are attenuated in the absence of Nkx2-5 in the chronic hypoxia model of PAH;

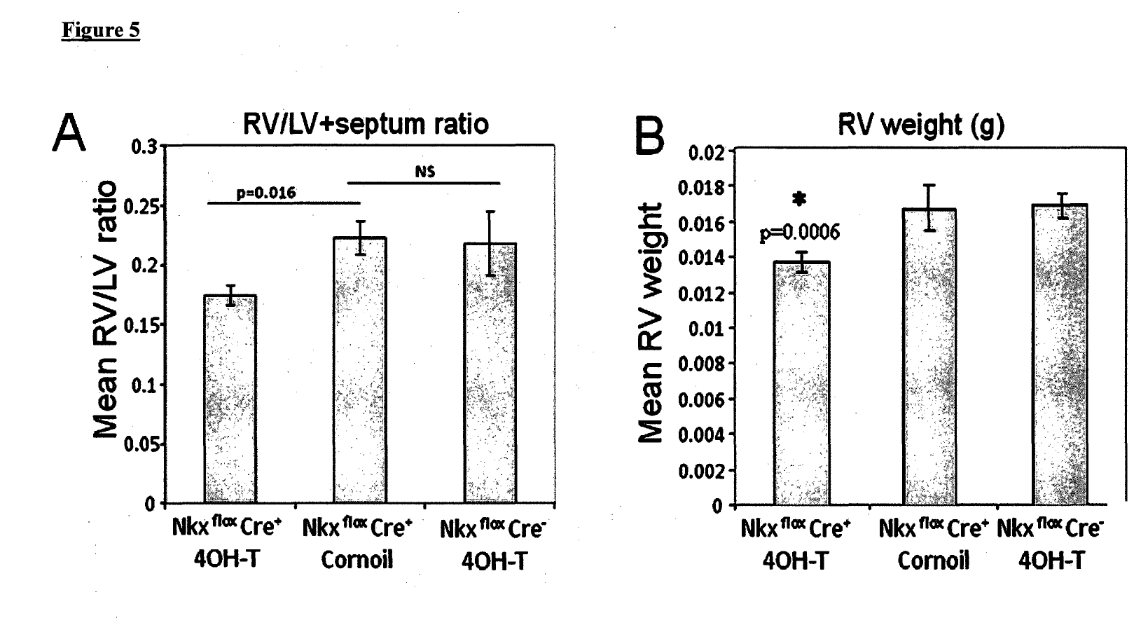

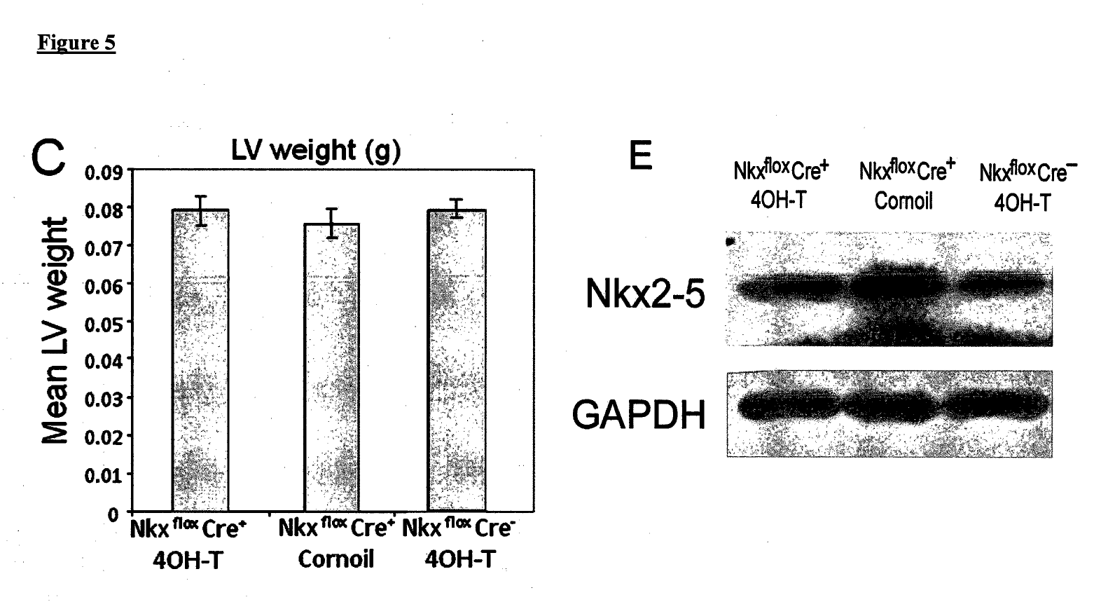

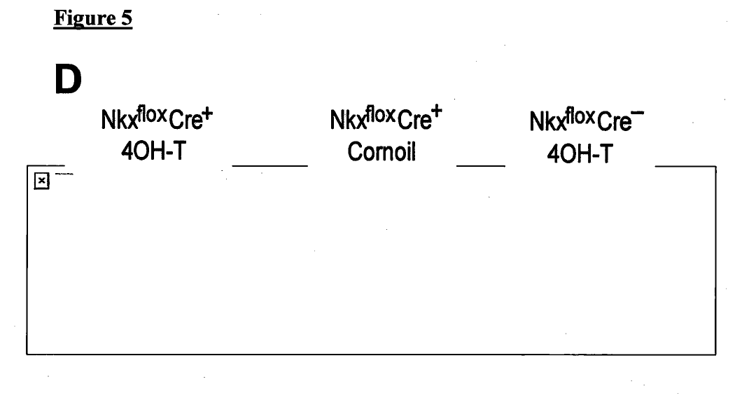

FIG. 5 shows the effect of conditional Nkx2-5 deletion on mouse heart in the chronic hypoxia model of PAH;

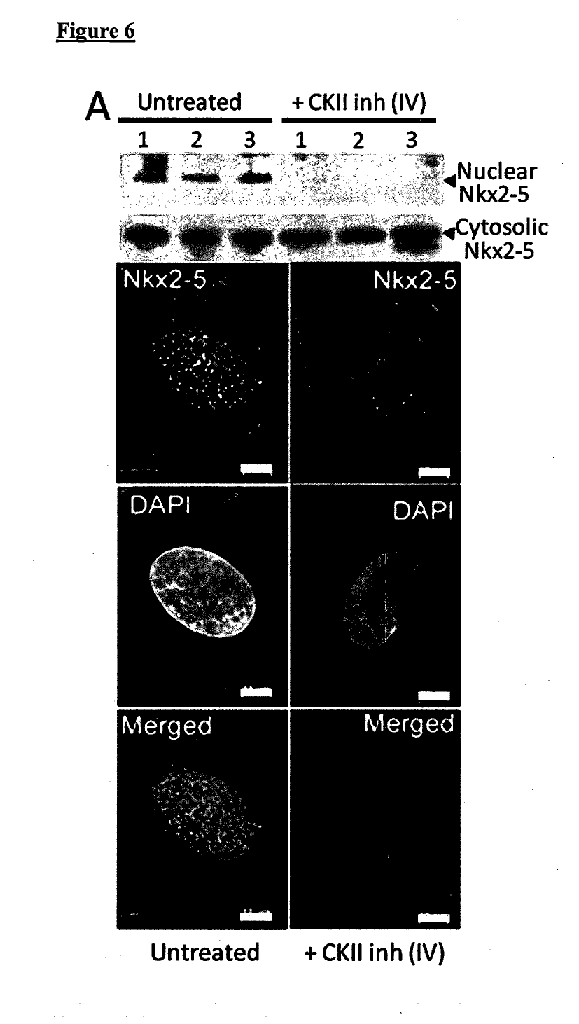

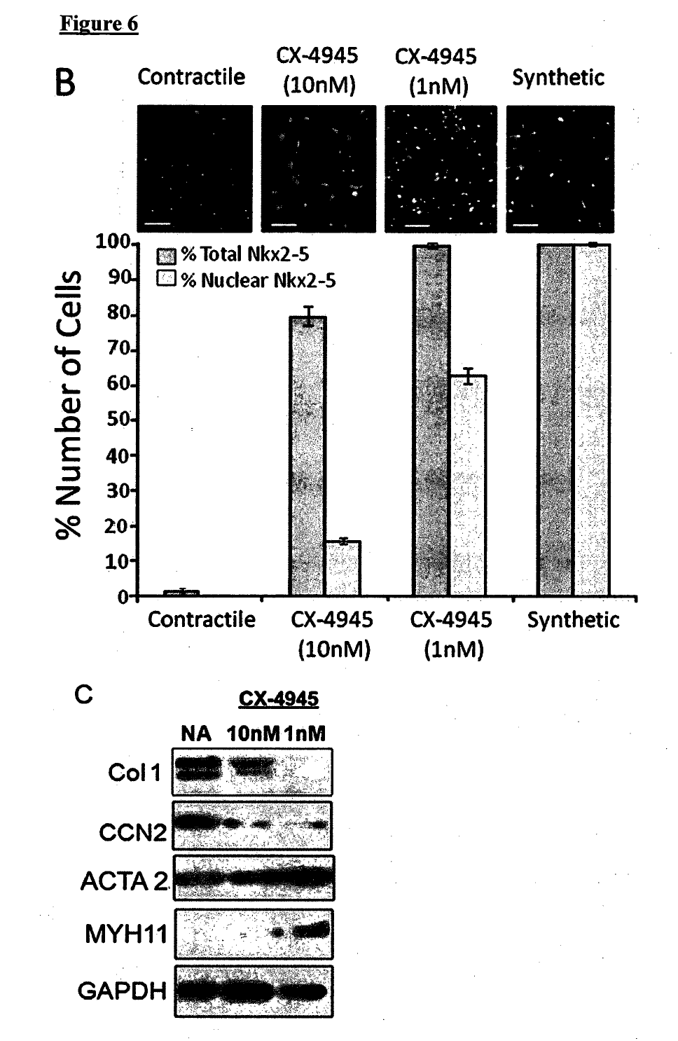

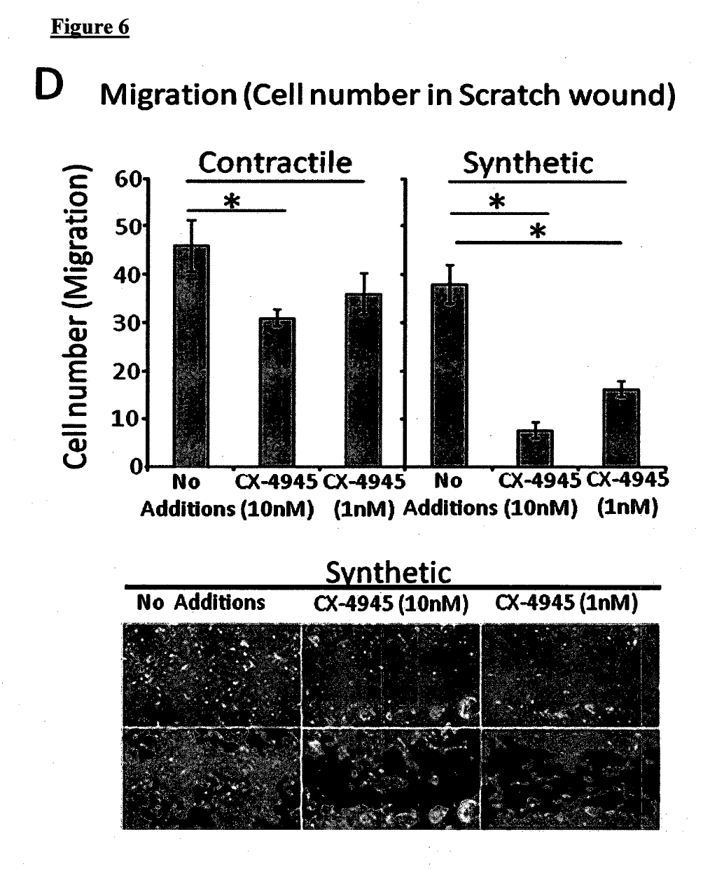

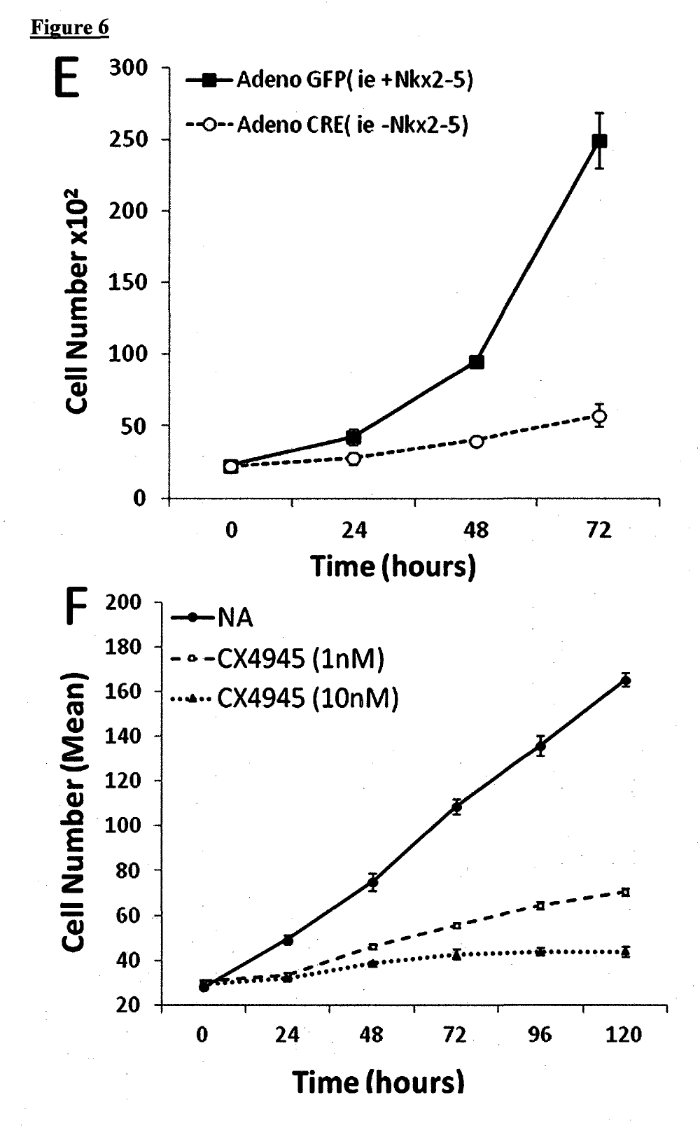

FIG. 6 shows CKII inhibition of Nkx2-5 nuclear translocation results in inhibition of phenotypic modulation, migration and proliferation of pulmonary arterial smooth muscle cells;

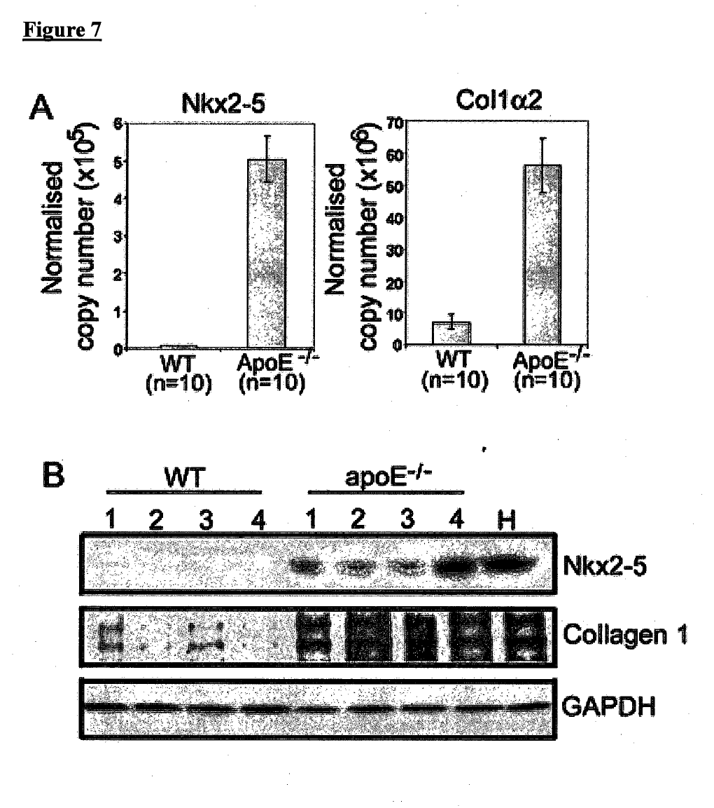

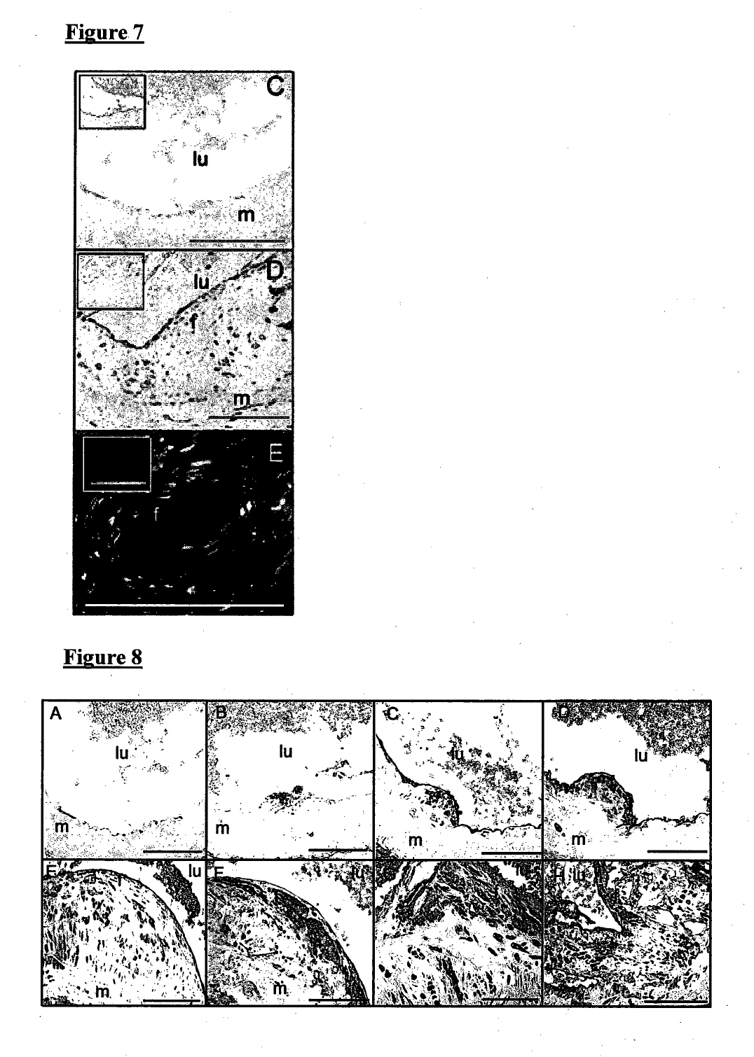

FIG. 7 shows that Nkx2-5 is expressed in atherosclerotic lesions of aortas of ApoE^{-/-} mice kept on an atherogenic diet but not in normal vessels;

FIG. 8 shows Nkx2-5 expression on developing atherosclerotic lesions in aortas of fat-fed apoE^{-/-} mice;

FIG. 9 shows expression of Nkx2-5 in collagen producing cells in the atherosclerotic lesions of fat-fed apoE^{-/-} mice;

FIG. 10 shows co-expression of Nkx2-5 in smooth muscle actin positive in the atherosclerotic lesions of fat-fed apoE^{-/-} mice;

FIG. 11 shows inhibition of Nkx2-5 in mice in vivo in the carotid ligation model of vascular remodelling results in a highly significant decrease of neointimal lesion formation;

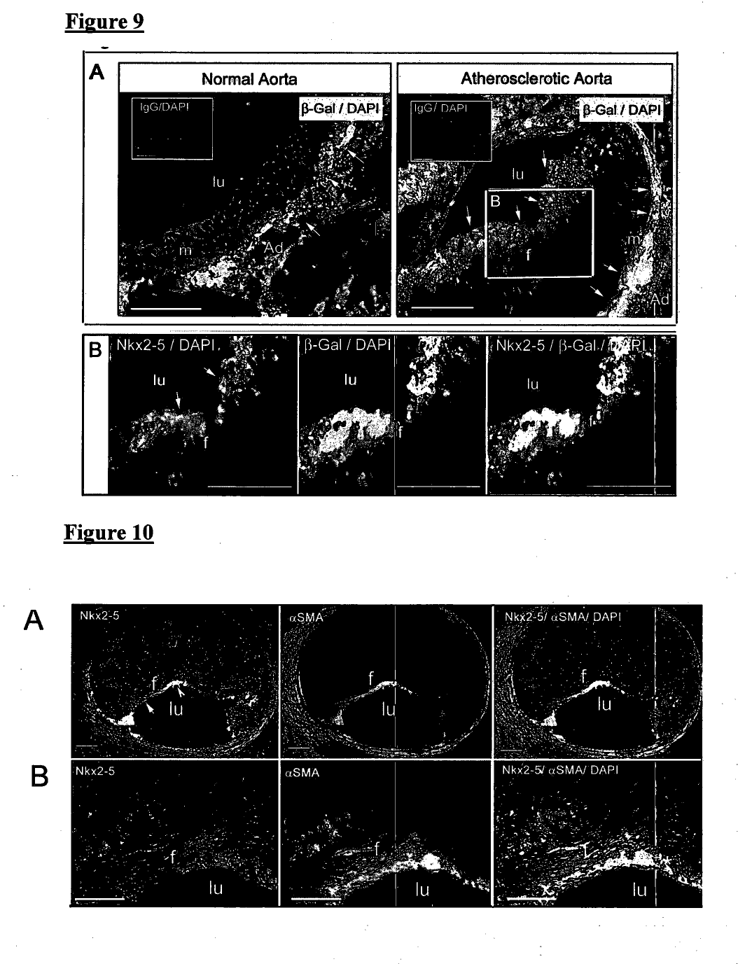

FIG. 12 shows Nkx2-5 expression in atherosclerotic lesions;

FIG. 13 shows SDS-PAGE and Western blot analysis of Nkx2-5 expression in human aortic smooth muscle cells undergoing phenotypic modulation in vitro;

FIG. 14 shows nuclear localisation of Nkx2-5 in ‘synthetic’ human smooth muscle cells;

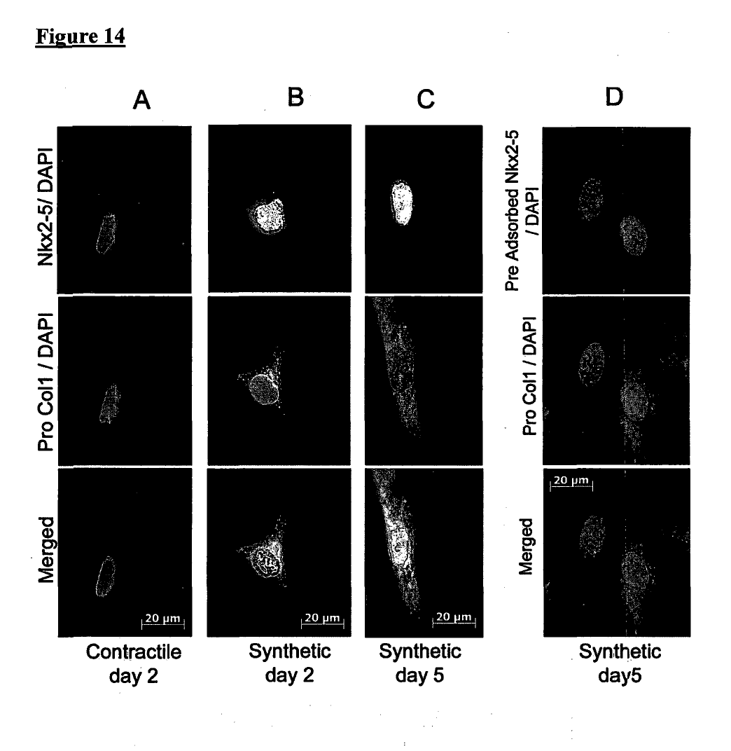

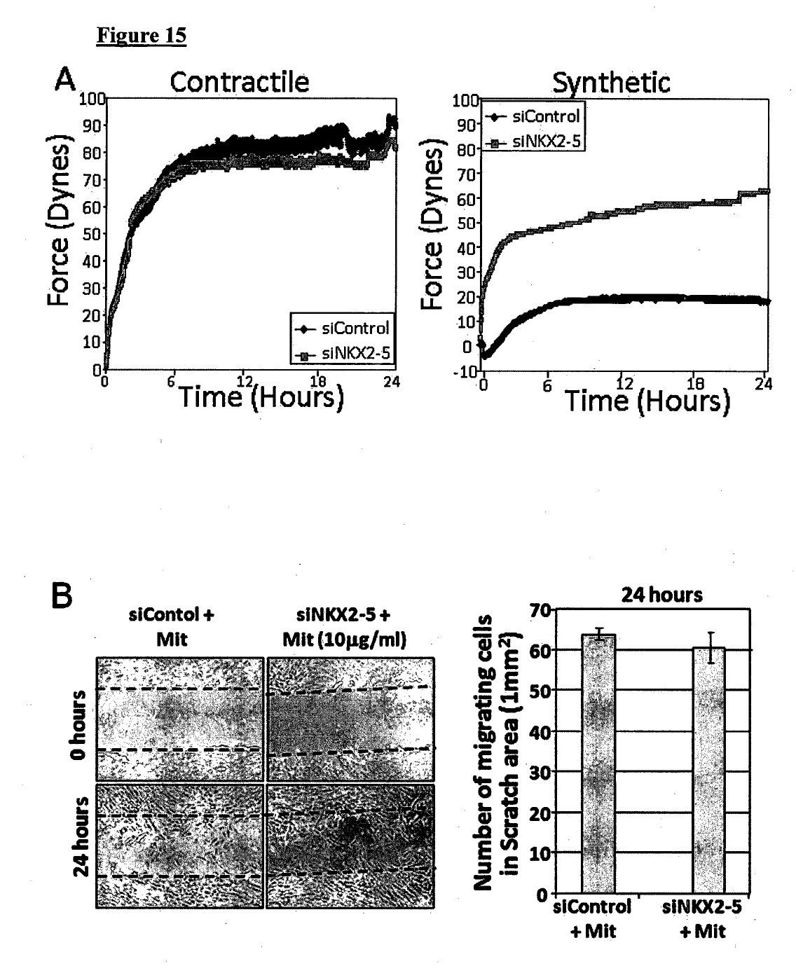

FIG. 15 shows that inhibition of Nkx2-5 results in a more contractile and less migratory phenotype;

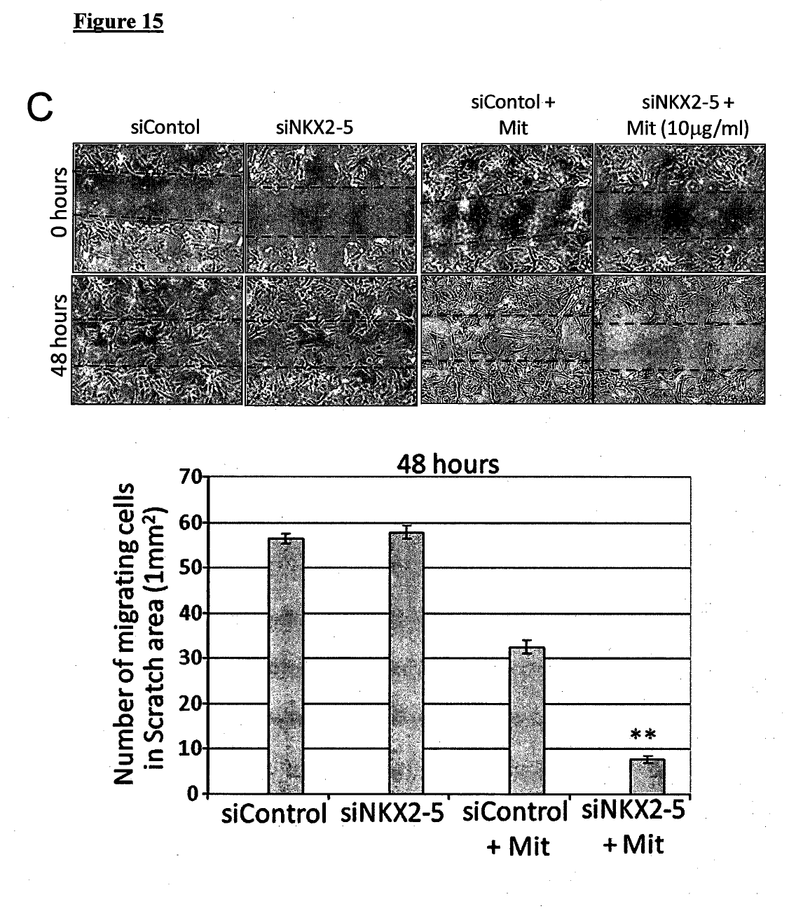

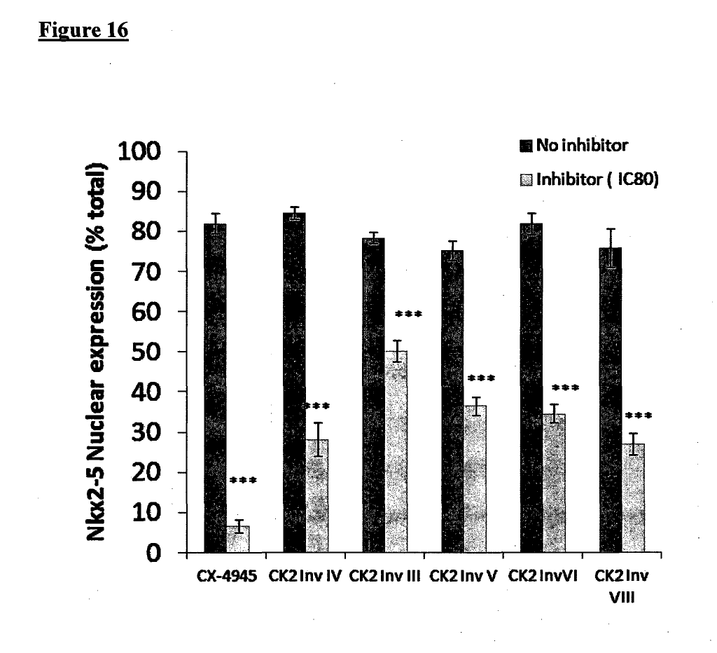

FIG. 16 shows the effect of CK2 antagonists on Nkx2-5 nuclear localisation;

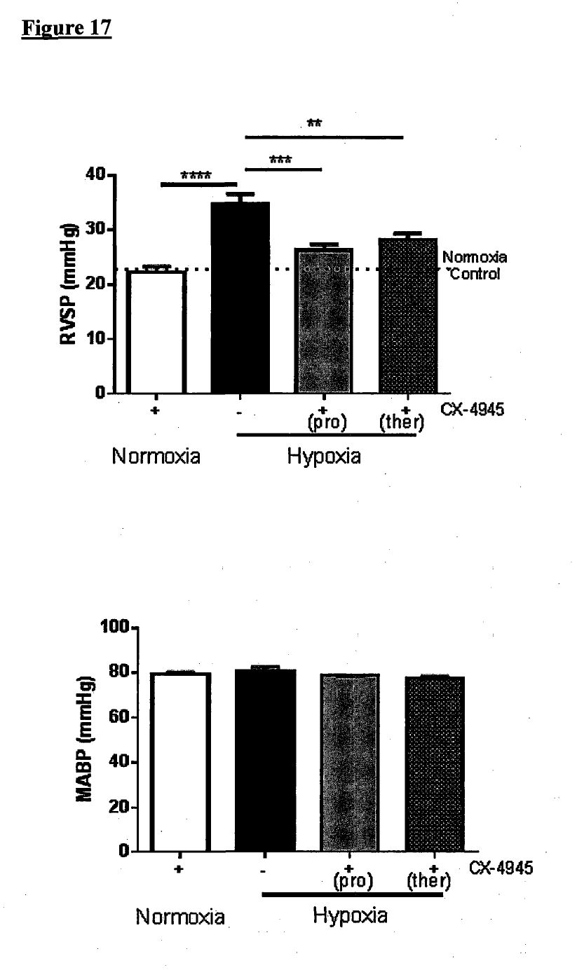

FIG. 17 shows the effect of CX-4945 antagonism on RVSP and systemic pressures in a pre-clinical model of PAH; and

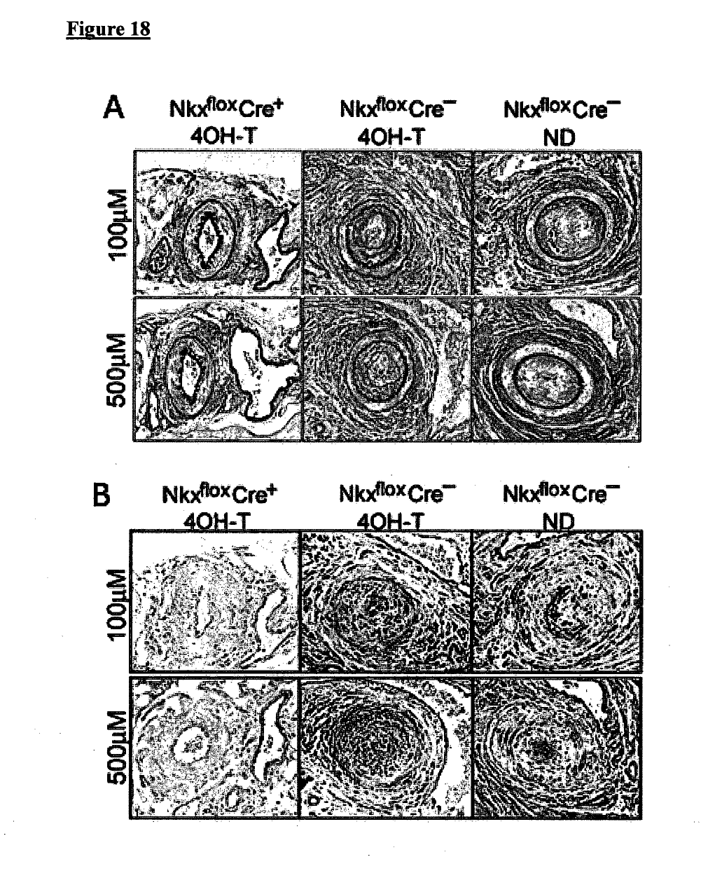

FIG. 18 shows the effect of Nkx2-5 deletion on vascular remodelling in the femoral artery ligation model.

### EXAMPLES

### Experimental Procedures

### Pulmonary Arterial Hypertension (PAH) Methods

Histology:

Paraffin-embedded lung tissue was sectioned and stained with hematoxylin and eosin, picro-sirius red (Collagen staining), or Van Geison stain (Elastin staining) reagents according to standard histological procedures. Pulmonary arterial muscularization was assessed by counting the number of vessels of less than 100 μm diameter in each lung section and defining them according to degree of muscularization.

Immunofluorescence:

Co-localization between cell-specific antigens was investigated using 2-, 3- and 4-colour immunofluorescence labelling. Fresh tissues were embedded in Cryo-M-Bed, and immediately snap-frozen in isopentane cooled by liquid nitrogen and subsequently stored at −70° C. before cryosectioning. Frozen sections of lung were used with specific primary antibodies and appropriate fluorescent tag-labelled secondary antibodies. Immunofluorescence was viewed and photographed on an Axioskop Z fluorescence microscope with an Axiocam digital camera in combination with Axiovision software. Specific antibodies for Nkx2-5, collagen type I, smooth muscle cell markers α-smooth muscle actin, smooth muscle myosin heavy chain, smoothelin), endothelial markers (e.g. CD31), stem cell markers (e.g CD34) were used in immunofluorescence experiments. Images were captured using a ×40 or ×20 Plan Neofluar lens and analyzed using KS400 software.

Hypoxia-Induced Pulmonary Hypertension:

The inducible conditional knockout mice Nkx2-5 flox Col1 CreERT mice were divided into 4 groups: (i) Hypoxia (10% oxygen) Nkx2-5^{flox}Cre⁺ with Tamoxifen induction, and the control groups (2) Hypoxia (10% oxygen) Nkx2-5^{flox}Cre⁺ with cornoil, (3) Hypoxia (10% oxygen) Nkx2-5^{flox}Cre⁻ with Tamoxifen and (4) normoxia (21% oxygen), and at least ten animals were used in each group. Tamoxifen/Cornoil was administered intra-peritoneally for five consecutive days starting at day 3. After a three week of exposure period to either normoxia or hypoxia, the animals were anaesthetised with 1.5% isoflourane and placed supine onto a heating blanket that was thermostatically controlled at 37° C. First the right jugular vein was isolated and a pressure catheter (Millar mouse SPR-671 NR pressure catheter with a diameter of 1.4 F, Millar Instruments, UK) introduced and advanced into the right ventricle to determine right ventricular systolic pressure (RVSP). As it is technically very difficult to insert the pressure catheter in the murine pulmonary artery, the RVSP is used as a surrogate for systolic pulmonary arterial pressure. Second, the mean arterial blood pressure (MABP) was measured by isolating the left common carotid artery and a pressure catheter introduced. Both RVSP and MABP were recorded onto a precalibrated PowerLab system (ADlnstruments, Australia). Animals were removed from the chamber individually and anesthetized immediately to minimize the time spent outside the hypoxic environment before hemodynamic measurement. The animals were then sacrificed, their hearts removed, and individual chamber weights were measured to evaluate RV hypertrophy (RV:LV+S ratio). First/second order pulmonary arteries were removed for vessel myography. The left lung was either cryopreserved, or fixed by inflation with 10% formalin in phosphate-buffered saline before paraffin embedding and sectioning.

Myography:

A wired small vessel myograph system was used to study the contraction and relaxation responses and vascular isometric tension measurements of contractile function. Phenylephrine (PE; 1 nM-50 mM) agonists were used to induce vasoconstriction in pulmonary arteries dissected from lungs of mice kept under normoxic or hypoxic conditions. The NO donor sodium nitroprusside (SNP; 1 nM-50 mM) was used to assess endothelial-independent relaxation. Vessels were studied at the same basal tension. After equilibration for at least 60 minutes, the vessel was challenged with Phenylephrine. Concentration-contraction response curves were constructed and expressed as a percentage of PE contraction or SNP relaxation. The concentration that causes 50% of the maximal contraction was estimated by fitting each concentration-response curve. Relaxation response to sodium nitroprusside was tested in ITAs precontracted with phenylephrine (1-3 mol/L) and expressed as a percentage of the precontraction value.

Cell Explantation, Primary Cells and Cell Culture:

Murine pulmonary artery smooth muscle cells were explanted from pulmonary arteries from adult mice which were maintained in culture as previously described. Briefly, arteries were harvested from anesthetized animals and placed in cold sterile PBS (pH 70.6). While still attached to the heart, the main pulmonary artery (PA) and right and left branch PAs were dissected and cleared of fatty tissue. The main PA was then dissected longitudinally. Endothelium was removed by gently scraping the luminal surface with a scalpel blade, and the adventitia was also removed from the vessel. The medial layer was minced using scalpel blades and incubated at 37° C. in M199 (GIBCO-BRL) supplemented with 0.1% collagenase I (Sigma) and 0.1% BSA (Boehringer-Mannheim) for 1 hour with gentle rotation. PASMCs were harvested by centrifugation and were routinely maintained in M199 containing 10% heat-inactivated FBS (GIBCO, Grand Island, N.Y.), 10 U/mL penicillin G sodium, 10 μg/mL streptomycin sulfate, 0.25 μg/mL amphotericin B, and 0.1 mg/mL gentamicin sulfate (GIBCO-BRL). Cells were passaged bytrypsinization using 0.05% trypsin/EDTA (GIBCO-BRL). Vascular SMCs were identified by their characteristic hill-and-valley morphology and immunohistochemical staining for α-smooth muscle actin. All experiments were performed in triplicate using cells between passages 2 and 4.

Migration, Gel Contraction and Fibrosis Assays:

The functional role of Nkx2-5 in cell migration and matrix contraction was assessed using well-established assays for migration (scratch wound), contraction (collagen gel contraction) and ECM production (qRT-PCR, Western blotting and immunofluorescence for fibronectin, collagen type I), to determine the role of Nkx2-5 in the different cell functions characteristic of fibrosis.

Hypoxia Sugen Protocol:

All animal procedures were conducted in accordance with the British Home Office regulations (Scientific Procedures) Act of 1986, UK. Animals were housed in a 12-hour light-dark cycle, food and water were accessible ad libitum. Adult female C57/B16 mice (at least n=5 per group, 20±2.5 g) received a weekly subcutaneous dose of SU5416 at 20 mg/kg, which was suspended in CMC (0.5% [w/v] carboxymethylcellulose sodium, 0.9% [w/v] sodium chloride, 0.4% [v/v] polysorbate 80, 0.9% [v/v] benzyl alcohol in deionized water). Three groups of animals were exposed to chronic normobaric hypoxia (10% O₂) in a ventilated chamber for 20 days. Control mice received only vehicle (5% DMSO, 40% PEG400, 55% dH₂O) by daily gavage, whereas treatment groups received CX-4945 in vehicle (80 mg/kg/daily) by daily gavage for either 20 days (prophylactic) or 13 days (therapeutic). Two groups of animals were kept in room air (normoxia) and received CX-4945 (80 mg/kg/daily) or vehicle for 20 days by daily gavage. At the end of the treatment period, mice were anesthetized and right ventricular systolic pressure (RVSP) and mean arterial blood pressure (MABP) was determined.

Haemodynamic Measurements:

Haemodynamic measurements of right ventricular systolic pressure (RVSP) and mean arterial blood pressure (MABP) were obtained from the animals after six weeks of hypoxia exposure and relevant drug treatment. The animals were anaesthetised with 1.5% isofluorane and placed supine onto a heating blanket that was thermostatically controlled at 37° C. First the right jugular vein was isolated and a pressure catheter (Millar mouse SPR-671 NR pressure catheter with a diameter of 1.4 F, Millar Instruments, UK) introduced and advanced into the right ventricle to determine RVSP. Second, MABP was measured by isolating the left common carotid artery and a pressure catheter introduced. Both RVSP and MABP were recorded onto a precalibrated PowerLab system (ADlnstruments, Australia).

### Atherosclerosis Methods

### Animals:

All animal work was performed under United Kingdom Home Office Licence and in accordance with all applicable laws and regulations. Apolipoprotein E knockout mice on a C57BL/6 background (apoE^{-/-}) and normal wild-type C57BL/6 were used in this study. In addition, Collagen type I α2 reporter transgenic mice (Col1α2-LacZ-Tg) were used. Col1 α 2-LacZ-Tg on the C57BL/6 background were bred with apoE^{-/-} animals to produce Col1 α 2-LacZ-Tg⁺apoE^{-/-} mice.

Cells:

Mouse Vascular smooth muscle cells were explanted from wild-type mouse aortas by an adapted procedure previously described. Commercially available human Aortic smooth muscle cells (Promocell) were used in invitro experiments. Vascular smooth muscle cells were grown to confluence for 48 hours. The post-confluent cells were then either cultured at high density in the absence of foetal bovine serum (conditions favouring the ‘contractile’ phenotype) for 2 to 8 days, or cultured at low density in the presence of 10% foetal bovine serum (conditions favouring the ‘synthetic’ phenotype.

Immunohistochemistry:

Tissues were post-fixed in neutral buffered formalin and paraffin embedded. Standard immunohistochemical protocols with antigen retrieval were performed with validated Nkx2-5 antibodies (Abcam, SantaCruz) or isotype-matched control antibody (Abcam, Santa Cruz). Specificity of the Nkx2-5 signal was also tested using antibody controls where the antibodies were pre-adsorbed with commercially available blocking peptide.

Immunofluorescence:

Fresh tissues were frozen and cryosections were cut, fixed in ice-cold acetone and used immunofluorescence labelling using standard protocols. In addition formalin fixed paraffin embedded samples were also used. Primary and isotype control antibodies were used followed by fluorescent-dye tagged secondary antibodies (Alexafluor, Invitrogen).

Antibodies:

The specificity of the polyclonal Nkx2-5 antibody (Santa Cruz) used in this study was validated using Western blot analysis. Appropriate immunoglobulin isotype control antibodies were used for each antibody as recommended by their manufacturers.

Image Analysis:

Images were captured using a ×40 or ×20 Plan Neofluar lens (NA and so forth) and analyzed using KS400 software (Imaging Associates, Bicester, UK). Lesion area was quantified using Image J software analysis. Statistical analysis was carried out using Prism software.

SDS-PAGE and Western Blotting:

SDS polyacrylamide gel electrophoresis (SDS-PAGE) was carried out on protein samples using Tris-glycine gels under denaturing conditions (Invitrogen) following standard protocols.

Quantitative Real Time-PCR:

Freshly harvested tissues cells were used for expression studies. Total RNA was isolated from mouse tissue using Trizol reagent (Invitrogen) or the RNeasy mini-kit (Qiagen) according to the manufacturer's instructions. RNA quality was confirmed using an Agilent 2100 Bioanalyzer (Agilent Technologies UK Ltd, UK). Reversed transcription was performed using the QuantiTect reverse transcription kit (Qiagen) according to the manufacturers' protocol. Quantitative PCR was performed using SYBR green-based assays (Quantace, UK) on a Rotorgene-6000 (Corbett Life Sciences, Australia). Four most stable reference genes were used to normalise as recommended by (GeNorm).

siRNA interference of Nkx2-5: siRNA for Nkx2-5 (ON-TARGETplus SMARTPool, 5′-CCTCAATCCCTACGGTTAT-3′ (SEQ ID No. 5); 5′-CCAACAACAACTTCGTGAA-3′ (SEQ ID No. 6); 5′-GCTACAAGTGCAAGCGGCA-3′ (SEQ ID No. 7); and 5′-CCGGGATTCCGCAGAGCAA-3′ (SEQ ID No. 8), and control off-target oligonucleotides (ON-TARGETplus Non-targeting Control pool (Dharmacon, USA) was transfected into vascular smooth muscle cells using Oligofectamine (Invitrogen) according to the manufacturers' instructions.

### A) Pulmonary Arterial Hypertension (PAH)

### Example 1

### NKX2-5 Expression in Human PAH

NKX2-5 is not expressed in the normal human pulmonary vasculature. However, it is expressed in the pulmonary vessels of patients with PAH. It is expressed in the media of large arteries (arrows, M) as well as in medium to small arteriols (pa) (FIG. 1A). NKX2-5 is expressed predominantly with smooth muscle cells as seen by co-localisation with a-smooth muscle actin (ACTA2, αSMA) expression in 3-colour immunofluorescence experiments on sections of human normal lungs or lungs from a patient with systemic PAH secondary to connective tissue disease (SSc-PAH) (FIG. 1B). Vascular pathology often includes phenotypic modulation of vascular smooth muscle cells where normal ‘contractile’ smooth muscle cells in the arterial media de-diferentiate to a more ‘synthetic’ phenotype which results in the upregulation of extracellular matrix genes such as collagen type I (COL1), Connective tissue growth factor (CCN2) and Fibronectin (FM) and the downregulation of contractile proteins such as smooth muscle myosin heavy chain (MYH11), α-smooth muscle actin (ACTA2) and smoothelin (SMTN).

Norman human pulmonary artery smooth muscle cells (HPASMC) can be cultured in vitro under conditions favouring the ‘contractile’ or the ‘synthetic’ state to mimic the progression from one state to the other thereby mimicing the phenotypic switching in vivo (FIG. 1C). Western blot analyses of HPASMC lysates shows cultured at contractile or synthetic conditions for 1 or 7 days, shows that NKX2-5 expression is very low in contractile cells but its expression increases as the cells progressively de-differentiate to a more ‘synthetic’ state in vitro (FIG. 1C). Representative blots of replicated experiments (n=6) are shown and GAPDH is used as a loading control. Similarly, HASMC grown in vitro under contractile or synthetic conditions where visualised using immunofluorescence (FIG. 1D). Contractile HASMC did not express NKX2-5, intracellular Pro Collagen Type I or FM but had normal ACTA2 fibres. However, in synthetic HASMC, NKX2-5 expression was clearly nuclear (white arrows), procollagen 1 and FM expression was up-regulated and ACTA2 expression was down-regulated and the ACTA2 fibrils were disorganised.

The inventors had previously demonstrated that NKX2-5 activated collagen 1α2 expression in smooth muscle cells ((2004) Mol Cell Biol. 24:6151-6161), and now tested the hypothesis that NKX2-5 has a major role in the phenotypic modulation of smooth muscle cells by inhibiting NKX2-5 expression using siRNA and looking at the expression of contractile and synthetic profile markers (FIG. 1E). Synthetic HASMC expressing NKX2-5 were untreated, treated with scrambled siRNA (siCON), or siNKX2-5 (50 nM or 250 nM). Knockdown of NKX2-5 resulted in down regulation of ‘synthetic’ phenotype COL1, CCN2 and FM. However, the expression of ‘contractile’ phenotype markers such as MYH11, ACTA2 and SMNT increased in the absence of NKX2-5. Representative blots of replicated experiments (n=3) are shown and GAPDH is used as a loading control.

### Example 2

### Conditional NKX2-5 Deletion in the Mouse Chronic Hypoxia Model of PAH

PAH is induced by chronic hypoxia which results in an increase in pulmonary artery pressure and vascular remodelling, consisting of thickening of the media layer of muscular arteries and muscularisation of distal vessels as well as a reduction of peripheral arteries. The condition is largely reversible however once normoxia has been restored. Chronic hypoxia-induced PAH has been modelled using mice and reproducibly demonstrates many the features that occur in humans such as remodelling resulting from pulmonary smooth muscle cell proliferation and hypertrophy and increased medial thickening that occurs soon after the onset of disease.

Inducible conditional null Nkx2-5 were created by mating Nkx2-5flox mice ((2004) Cell. 117:373-386) with Collagen type Iα2 enhancer-driven CreERT mice. The inventors had previously generated transgenic mice in which a mesenchemal cell-specific enhancer of the mouse Co1α2 gene directs expression of the polypeptide consisting of a fusion between Cre recombinase and a mutant ligand-binding domain of the estrogen receptor ((1997) Biochem Biophys Res Commun. 237: 752-7) (FIG. 2A). In these Col1α2 Cre-ERT-transgenic mice, administration of Tamoxifen (4-OHT) activates Cre recombinase in cells where Collagen type Iα2 is being over-expressed such as fibroblasts and vascular smooth muscle cells ((2002) Am J Pathol. 160: 1609-17). Polymerase chain reaction using specific primers (P1-P2 and P1-P3) were used to ascertain whether recombination of the loxP sites has occurred and therefore whether Nkx2-5 has been deleted. Nkx2-5 was knocked out in adult Nkx2-5^{flox} Col1α2CreERT+(Nkx2-5^{fox} Cre⁺) mice in remodelling vessels, by Tamoxifen (4-OHT) administration for 5 consecutive days beginning on day 3 after the mice were placed in the hypoxic chamber (FIG. 2B, C, D).

Two groups of control mice were used: Nkx2-5^{flox}Col1α2CreERT⁺ injected with cornoil instead of tamoxifen and Nkx2-5^{flox}Col1α2CreERT⁻ injected with tamoxifen. After 21 days in hypoxia the mice were removed and sacrificed. Mice treated in the identical way were also kept under normoxic conditions in paralel. Pulmonary arteries from these mice were analysed for Nkx2-5 protein expression by western blotting (FIG. 2B). Representative blots of replicated experiments (n=12) are shown and GAPDH is used as a loading control. Total RNA expression was also measured using quantative PCR (qPCR) (FIG. 2C). Finally, sections of lungs from these mice were immunodecorated with a specific Nkx2-5 antibody and visualised using a fluorescent dye (Alexa 488) (FIG. 2D). The same results were obtained using all three methods analysis. No Nkx2-5 was detected in pulmonary vessels under normoxic conditions or in Nkx2-5 null mice (Nkx2-5^{flox}Col1α2CreERT⁺ treated with tamoxifen) under hypoxic conditions. However, Nkx2-5 expression was detected and elevated in hypoxic control mouse groups ie Nkx2-5^{flox}Col1 α 2CreERT⁺ injected with corn oil instead of tamoxifen and Nkx2-5^{flox}Col1 α 2 CreERT⁻ injected with tamoxifen.

### Example 2

### Effect of Conditional Nkx2-5 Deletion on Pulmonary Vessels

Muscularisation of small (20-50 μm), medium (40-70 μm) and large (greater than 70 μm) arteries occurs after 21 days under hypoxic conditions in both control mice groups (Nkx2-5^{flox}Col1 α 2CreERT⁺ injected with corn oil and Nkx2-5^{flox}Col1 α 2CreERT—administered with tamoxifen). However, the Nkx2-5 null mice under hypoxic conditions show significantly less vascular muscularisation (p<0.00005) in small, medium and large vessels (FIGS. 3A and B). Under normoxic conditions the vessels walls of small, medium and large vessels resemble the size and thickness of the vessels found in the lungs of the Nkx2-5 null mice under hypoxic conditions (FIGS. 3A and B).

Immunohistochemistry with α-smooth muscle actin antibody was carried out on sections of the entire left lobe of the lungs of mice thus identifying the media of each vessel. The circumference and medial thickness of the all the vessels in the entire section were measured (n=5 mice per group and more than 250 vessels per group) quantified (FIG. 3B). Representative images of different sized vessels are shown in (FIG. 3A).

At the end of 21 days of hypoxic treatment the mice were immediately anaesthetised and their right ventricular systolic pressure (RVSP) was measured using a pressure catheter. Under normoxic conditions the RVSP is 20±1.4 mmHg. After 21 days in hypoxia the RVSP of the control groups (Nkx2-5^{flox}Col1 α 2CreERT⁺ injected with cornoil and Nkx2-5^{flox}Col1 α 2CreERT⁻ administered with tamoxifen) was elevated to 40.18±2.44 mmHg and 37.6±1.83 mmHg respectively. However the RVSP of the Nkx2-5 null mouse group (Nkx2-5^{flox}Col1 α 2 CreERT⁺ treated with tamoxifen) was significantly lower at 29.15±2.3 mmHg (p=0.0076). There was no difference in mean arterial blood pressure (MABP) in any of the groups (data not shown).

Finally, myograghy was carried out on first and second order pulmonary arteries dissected from the lungs of the mice after the RVSP measurements were taken. A wired small vessel myograph system was used to study the contraction and relaxation responses by making vascular isometric tension measurements to assess contractile function (FIG. 3D). Phenylephrine (PE; 1 nM-50 mM) was used to induce vasoconstriction, and the NO donor sodium nitroprusside (SNP; 1 nM-50 mM) was use to induce relaxation. The maximal contraction response curves of vessels from Nkx2-5 null mouse group (Nkx2-5^{flox}Col1 α 2 CreERT⁺ treated with tamoxifen, solid line) have shifted to the left suggesting that the vessels are more sensitive to PE and SNP and therefore they are less stiff and more compliant than the vessels from the control groups (dashed line).

### Example 4

### Effect of Nkx2-5 Deletion on Pulmonary Collagen Levels

Pulmonary collagen levels are attenuated in the absence of Nkx2-5 in the chronic hypoxia model of PAH. Representative lungs (n=8) isolated from mice placed in chronic hypoxia (10% oxygen) for 21 days stained with Hematoxilin and Eosin (H & E) (FIG. 4A), Picrus Sirus Red (PSR) for collagen (FIG. 4B), and viewed under polarised light (PSR-PL) were nascent collagen shows as yellow/green (FIG. 4C). Densitometric quantification of images of the left lobes of hypoxic lungs (n=8) stained with Picrus Sirus Red stain and visualised using polarised light (FIG. 4E) showed a significant decrease of collagen levels in the lungs of Nkx2-5 null mice compared to the control groups. These findings were also supported by collagen type I protein levels found in hypoxic lung homogenates analysed by SDS PAGE, western blot and immunodecorated using Col1 specific antibodies. GAPDH levels were used as loading control. Lung homogenates form three mice of each group are shown (FIG. 4E).

### Example 5

### Effect of Conditional Nkx2-s Deletion on Cardiac Hypertrophy

Right ventricular (RV) hypertrophy is a well described characteristic of the chronic hypoxia model of PAH. Conditional Nkx2-5 deletion after 21 days of hypoxia reduced RV hypertrophy as observed by the significant decrease in RV/LV+S ratio (FIG. 5A) and the individual wet weights of the right ventricles alone (FIG. 5B). There were no significant changes in left ventricle weight across the groups (FIG. 5C). The inventors tested whether this decrease is a result of the deletion of Nkx2-5 from the cardiac tissues or whether it is due to pulmonary changes by assessing the expression of Nkx2-5 in the mouse hearts. Immunohistochemistry using specific Nkx2-5 antibodies revealed that Nkx2-5 was not knocked down in heart tissues (FIG. 5D). This can be explained by the fact that the collagen type I enhancer that drives expression of the Cre recombinase is not activated in hearts of mice placed in hypoxic chambers for 21 days although it is activated in the lungs and pulmonary vessels (data not shown). SDS-PAGE and western blot analysis of Nkx2-5 expression in right ventricle homogenates also reveals no significant differences between the groups (FIG. 5E).

### Example 6

### Pharmacological Regulation of Nkx2-5

Nkx2-5 is regulated by cytoplasmic-nuclear translocation, a process regulated in part by casein kinase II-dependent phosphorylation and results in increased DNA-binding activity.

Nuclear translocation of Nkx2-5 was abolished in vitro in three human pulmonary smooth muscle cell lines by a comercially available CKII inhibitor although cytosolic expresion was unaffected (FIG. 6A). Another CKII inhibitor (CX-4945), can inhibit de-differentiation of human pulmonary smooth muscle cells from contractile to synthetic by inhibiting Nkx2-5 nuclear translocation in vitro in a dose dependent manner with an effective concentration of 1 nM (FIG. 6B). Western blot analysis of human pulmonary cell lysates treated with CX-4945 at 1 nM and 10 nM reveals a down regulation of ECM protein expression COL1 and CCN2 and an upregulation of contractile protein expression (ACTA2, MYH11) confirming the inhibitory effect of CX-4945 on the process of phenotypic modulation (FIG. 6C). Nkx2-5 expression in vascular smooth muscle cells also results in a pro-migratory synthetic phenotype. The effect of CX-4945 on human pulmonary smooth muscle cells in a standard scratch assay of migration in the presence of mitomycin (to abolish proliferation) is inhibitory in a dose dependent manner and reveals that CKII inhibition prohibits the de-differentiation of smooth muscle cells (FIG. 6D). Vascular smooth muscle cell from arteries of Nkx2-5^{flox}Col1 α 2 CreERT⁺ were explanted and cultured in vitro under conditions favouring the synthetic phenotype and express Nkx2-5. Each cell line was then infected with Adenovirus harbouring Cre recombinase (or control Adenovirus) to delete Nkx2-5. Cell proliferation was measured in the presence/absence of Nkx2-5. The absence of Nkx2-5 resulted in a significant decrease in the rate of proliferation (FIG. 6E). In addition, the proliferation of human pulmonary smooth muscle cells that were grown under conditions favouring Nkx2-5 expression (synthetic), was measured in the presence/absence of CX-4945 (10 nM and 1 nM). There was a highly significant dose-dependent decrease in the proliferation rate of over 5 days (FIG. 6F).

### B) Atherosclerosis

### Example 7

### Nkx2-5 is Expressed in Atherosclerotic Aortas of apoE Knockout Mice

The inventors used the apoE^{-/-} mouse model of atherosclerosis to examine whether Nkx2-5 was expressed in atherosclerotic lesions. Aortas were obtained from wild-type (n=10) and apoE^{-/-} mice (n=10) fed on a high cholesterol diet. Total RNA and protein were extracted from the aortas of these mice and analysed by quantative PCR and SDS-PAGE western blotting respectively (FIG. 7). The results show that aortas from apoE^{-/-} mice express Nkx2-5 mRNA (FIG. 7A) and protein (FIG. 7B) whereas there is no detectable expression in aortas from wild-type mice. Collagen type I levels are also elevated in the apoE^{-/-} aortas.

Furthermore, immunohistochemical staining using a specific Nkx2-5 antibody confirms that Nkx2-5 is expressed within aortic atherosclerotic lesions where as it is absent from normal wild type vessels (FIG. 7C). Specifically, high Nkx2-5 staining is observed within the fibrous cap although some Nkx2-5⁺ cells are scattered in the tunica media and throughout the lesion (FIG. 7D). No Nkx2-5⁺ cells are observed in wild-type vessels (FIG. 7C). Immunofluorescence double-labelling of apoE^{-/-} aortic lesions revealed that Nkx2-5 co-localised with the nuclear stain 4,6-diamidino-2-phenylindole (DAPI) (FIG. 7E).

### Example 8

### Expression of Nkx2-5 During Atherosclerotic Plaque Progression

There is no detectable expression of Nkx2-5 in normal non-lesional vessels (FIG. 8A), although Nkx2-5⁺ cells are seen from the earliest stages of intimal thickening and fatty streak formation in the apoE null mice (FIG. 8B). As the fatty streak expands, so Nkx2-5 expression becomes more widely distributed (FIGS. 8C and D) until a large majority of the cells appear to be positive (FIG. 8E). With the development of the complex lesion with a well defined fibrous cap, Nkx2-5 expression becomes particularly focussed to the cap (FIGS. 8F, G). Finally, in late stage highly complex lesions, although Nkx2-5 expression is maintained in fibrous cap it appears to be slightly decreased (FIG. 8H).

### Example 9

### Expression of Nkx2-5 by Collagen-Producing Cells

To test the hypothesis that the Nkx2-5 expressing cells within the atherosclerotic lesions produce collagen, the inventors performed immunofluorescence labelling experiments on atherosclerotic lesions in Col1α2-LacZ⁺apoE^{-/-} mice fed a normal or a high fat diet using specific antibodies to Nkx2-5 and β-galactosidase β-Gal) transgene to indicate expression of the collagen Iα2 transgene (FIG. 9). In atherosclerotic aortas taken from fat-fed Col1α2-LacZ⁺apoE^{-/-} mice, cells expressing β-Gal were present in the adventitia and within the lesion, primarily localised within the fibrous cap (FIG. 9A right panel). In the lesion-free aorta, expression of β-Gal was low and confined to cells within the adventitia (FIG. 9A left panel). No staining was observed using a isotype control antibodies (FIG. 9A, insets). Detailed examination of cells within the lesion under higher magnification using specific antibodies for Nkx2-5, β-Gal and DAPI reveal that Nkx2-5 co-localises with β-Gal in the fibrous cap (merged) confirming Nkx2-5⁺ cells also express the Col1α2 transgene (FIG. 9B).

Quantification of immunofluorescence staining of Nkx2-5⁺ cells within lesions in vessels (n=16) which were also positive for β-Gal (Nkx2-5⁺ β-Gal⁺) show 43.98±5.46% of the total cells within the lesion, excluding the media, are Nkx2-5⁺ β-Gal⁺ (Table 1). Moreover, the difference between Nkx2-5⁺ (52.8±3.12%) and Nkx2-5⁺ β-Gal⁺ (43.98±5.46%) is not statistically significant (p<0.27) (Table 1). These data strongly support the notion that Nkx2-5⁺ cells in the lesion produce collagen type I. The inventors also performed four-colour immunofluorescence to determine whether the Nkx2-5⁺ collagen-producing cells also express α-smooth muscle actin (αSMA⁺) (data not shown). The inventors used specific antibodies for Nkx2-5, β-Gal and αSMA as well as DAPI.

Appropriate isotype controls for each antibody were carried out for all immunofluorescence labelling in this study and subsequent studies described below, both individually and in combination and were all negative. Quantification of lesional staining (n=16) shows that 31.4±3.47% of the total cells within the lesion are Nkx2-5⁺ β-Gal⁺εSMA⁺ and the difference between Nkx2-5⁺ (52.8±3.12%) and Nkx2-5⁺ β-Gal⁺α(SMA+(31.4±3.47%) is statistically significant (p<0.005) (Table 1). The data suggest that the majority of the Nkx2-5⁺ cells within the atherosclerotic lesion are collagen-producing cells and most, but not all, of these express the smooth muscle cell marker, αSMA.

TABLE 1
Quantification of immunofluorescence staining of
Nkx2-5⁺ cell populations in atherosclerotic lesions

| | | Positive | | |
|---|---|---|---|---|
| | Number | cells | | |
| | of lesions | (% of | | |
| Gene Expression | (n) | Total) | SEM | p value |
| Nkx2-5⁺ | 24 | 52.8 | 3.1 | — |
| Nkx2-5⁺/Col1a2-LacZ-Tg | 16 | 44.0 | 5.5 | 0.27 |
| Nkx2-5⁺/α-SMA⁺/Col1a2- | 16 | 31.5 | 3.5 | 0.005 |
| LacZ-Tg | | | | |
| Smooth muscle cell | | | | |
| Nkx2-5⁺/α-SMA⁺ | 16 | 40.1 | 0.8 | 0.028 |
| Nkx2-5⁺/Smtn⁺ | 12 | 30.5 | 1.9 | 0.0004 |
| Nkx2-5⁺/sm-MHC⁺ | 12 | 31.8 | 3.2 | 0.002 |
| Nkx2-5⁺/SMC⁺(α- | 24 | 34.1 | 2.0 | 0.0002 |
| SMA⁺, Smtn⁺, sm-MHC⁺) | | | | |
| Macrophage | | | | |
| Nkx2-5⁺/F480⁺ | 8 | 1.0 | 0.6 | <0.0001 |
| Endothelial | | | | |
| Nkx2-5⁺/CD31⁺ | 8 | 7.4 | 2.1 | <0.0001 |
| Nkx2-5⁺/Flk1⁺ | 12 | 18.8 | 3.5 | 0.0002 |
| Stem cell, progenitor cell | | | | |
| Nkx2-5⁺/Isl1⁺ | 12 | 1.34 | 0.7 | <0.0001 |
| Nkx2-5⁺/Flk1⁺/Isl1⁺ | 12 | 0.5 | 0.3 | <0.0001 |
| Nkx2-5⁺/CD34⁺ | 12 | 3.3 | 1.4 | <0.0001 |
| Nkx2-5⁺/cKit⁺ | 12 | 0.5 | 0.3 | <0.0001 |

*P values: Student's T-test, compared with Nkx2-5⁺ population.

### Example 10

### Expression of Nkx2-5 in Smooth Muscle Cells

In order to fully investigate whether the type of Nkx2-5 expressing cells within the atherosclerotic lesion are vascular smooth muscle (SMC), the inventors performed immunofluorescence triple-labelling experiments in the atherosclerotic lesions from apoE^{-/-} mouse aorta. Antibodies for α-SMA were used in conjunction with Nkx2-5 (FIG. 10) and the nuclear stain DAPI. To gain an overall appreciation of the regional patterns of Nkx2-5 staining, a low power magnification of a transverse section through the entire lesional vessel was examined (FIG. 10 A). Nkx2-5 expression co-localises with αSMA in the fibrous cap and shoulder region of lesion although there are individual Nkx2-5⁺ αSMA⁺ cells within the media (FIG. 3A). At higher magnification (FIG. 10B), a high degree of co-localisation between nuclear Nkx2-5 and cytoplasmic αSMA within the fibrous cap is apparent. Quantificational analysis of Nkx2-5⁺ staining in the lesions (n=24) but not in the media of different vessels (Table 1) shows that 52.8±3.12% of the total cells within the lesion are Nkx2-5⁺.

The number of total cells in the lesion (excluding the media) in a particular field of view was quantified. Nkx2-5⁺ αSMA⁺ cells comprise 40.1±0.79% of the total cells within the lesion excluding the media. The apparent decrease between Nkx2-5⁺ and Nkx2-5⁺ αSMA⁺ cells is statistically significant (p<0.028) and suggests that not all Nkx2-5⁺ within the lesion also expressed αSMA. Similar data were obtained using Nkx2.5 and the other smooth muscle cell markers including sm-MHC and Smtn (data not shown). Nkx2-5 co-localises with sm-MHC and Smtn in cells located within the lesion. Nkx2-5⁺/sm-MHC⁺ and Nkx2-5⁺/Smtn⁺ cells are observed inside the core and fibrous cap of the lesion as well as within the medial layers of the vessel. Quantification to calculate the frequency of Nkx2-5⁺ sm-MHC⁺ and Nkx2-5⁺ Smtn⁺ cell populations within aortic lesions (n=24), excluding the vessel wall, shows that 30.5±1.94% of the total cells within the lesion are Nkx2-5⁺/Smtn⁺ and 31.8%±3.17 are Nkx2-5⁺/sm-MHC⁺ (Table 1). The difference between Nkx2-5⁺ and Nkx2-5⁺/Smtn⁺ or Nkx2-5⁺/sm-MHC⁺ cells expressed as a percentage of total cells within the lesion is statistically significant (p<0.0004 and p<0.002) again that not all Nkx2-5⁺ within the lesion are expressing SMC markers.

### Example 11

### Expression of Nkx2-s in Other Cell Types within the Atherosclerotic Lesions

The inventors investigated whether other cell types within the atherosclerotic plaques express Nkx2-5. No co-expression was detected with the macrophage marker F4/80, suggesting that the Nkx2-5⁺ cells in the lesion are not of the myeloid lineage (data not shown). However, the inventors tested for endothelial CD31 (platelet-endothelial cell adhesion molecule) expression within the lesions using immunofluorescence labelling. Unexpectedly, the data revealed the presence of Nkx2-5⁺ CD31⁺ cells within the lesions. The inventors quantified the staining in different atherosclerotic vessels (n=8) and found that 7.41±2.13% of the total cells within the lesion are Nkx2-5⁺CD31⁺ (data not shown and Table 1).

Recent reports describe multipotent cardiovascular progenitor stem cell populations which are Nkx2-5⁺. These include embryonic stem cell-derived populations with the signatures Isl1+/Nkx2-5⁺/Flk1⁺ and Nkx2-5⁺/c-Kit⁺ which can give rise to cells of cardiac, endothelial and vascular smooth muscle lineages and cells expressing the hematopoietic bone-marrow derived precursors marker CD34. Their potential role was examined, by investigating their frequency within atherosclerotic lesion. Immunofluorescence labelling using specific antibodies for Nkx2-5, Flk-1 (VEGF-R2), Isl1 (Islet-1) and DAPI (data not shown), showed very few Isl1⁺/or Nkx2-5⁺/Isl1⁺ cells within the lesion (<1.4±0.72%). However, there is a significant Nkx2-5⁺/Flk1⁺ population. Quantification of atherosclerotic lesions (n=12) revealed that 18.8±3.5% of the cells are Nkx2-5⁺ Flk-1⁺ (Table 1). Nkx2-5⁺/Flk1⁺/Isl1⁺ cells within atherosclerotic lesions were not present. Immunofluorescence labelling using specific antibodies for Nkx2-5, c-Kit and DAPI (Table 1) shows that there are c-Kit⁺ cells in the lesions, but these cells do not co-express Nkx2-5. Finally, the inventors detected a very small population (3.3±1.42%) of lesional cells that co-expressed Nkx2-5 and CD34 (Table 1).

### Example 12

### NKX2-5 Knockdown in Neointimal Lesions in the Carotid Ligation Model of Vascular Remodelling

Carotid ligation allowed the inventors to induce the formation of a neointimal lesion in the mouse carotid artery. Inducible conditional null Nkx2-5 were created by mating Nkx2-5flox mice ((2004) Cell. 117:373-386) with Collagen type Iα2 enhancer-driven CreERT mice (see FIG. 2A). In these Col1α2 Cre-ERT-transgenic mice, administration of Tamoxifen (4-OHT) activates Cre recombinase in cells where Collagen type Iα2 is being over-expressed such as fibroblasts and vascular smooth muscle cells. The mice were anaesthetised, their common carotid arteries were exposed and ligated as described in. The Nkx2-5^{flox}Col1α2CreERT⁺ (Nkx2-5 fox Cre⁺) mice were put on either a normal rodent diet (control group) or a normal rodent diet containing Tamoxifen (4-OHT at 400 mg/kg, ((2007) Genesis 45:11-16) (experimental group). The mice were allowed to recover for 28 days prior to sacrifice. The carotid arteries were processed, paraffin-embedded and sectioned from the ligature at 100 μM intervals. Nkx2-5 was knocked out in adult Nkx2-5^{flox}Col1α2CreERT⁺ (Nkx2-5^{flox} Cre⁺) mice in the remodelled carotid arteries of tamoxifen-fed mice but not control mice as shown be immunohistochemical staining on sections taken at 500 μM from the ligature using an Nkx2-5 specific antibody or isotype control (IgG). Two representative mice of each group are shown, and a clear decrease in neointimal lesion production was observed (FIG. 11A).

In addition, all the sections from both control (n=n) and experimental (n=10) mouse groups taken along the carotid artery at 100 mM intervals, were stained with hematoxylin and eosin and analysed for maximum plaque area using Image J. Statistical analysis (2-way ANOVA) was carried out with distance along the carotid and treatment (4-OHT administration) as the two variables. It showed that distance along the carotid was not a significant determining factor for lesion size, but that treatment was. Therefore, overall, there were smaller lesions in the tamoxifen-treated animals than in controls (−46.9%, p<0.0001) and Nkx2-5 inhibition has an extremely significant effect on lesion size (treatment accounts for 59.75% of the total variance, F=26.18. DFn=21 DFd=357) (FIG. 11B).

### Example 13

### NKX2-5 in Human Atherosclerotic Lesions

Nkx2-5 is expressed in human atherosclerotic lesions (FIG. 12). NKX2-5 expression is observed in lesions in the aorta (data not shown), the left coronary artery (data not shown), the peripheral vasculature (FIG. 12 A) and carotid artery (FIG. 12B). An occluded and partly calcified popliteal artery from an amputated limb of a patient with peripheral arterial disease (FIG. 12 A) and tissue removed from carotid endarterectomy (FIG. 12 B) were stained with Van Geisen-elastin stain (VG-E, purple), Masson's Trichrome for extracellular matrix (MT, blue for collagen) and immunostained with specific antibodies for a-smooth muscle actin (α-SMA) and Nkx2-5. Expression of Nkx2-5 co-localised with that of α-SMA in the media, confirming its expression by vascular smooth muscle cells. Expression is also observed in the fibrous cap, the shoulder as well as in cells inside the neointimal lesion. Expression is also observed in the walls of smaller arterioles (insets FIGS. 12A &B).

### Example 14

### Nkx2-5 in Phenotypic Modulation of Human Arterial Smooth Muscle Cells

Commercially available human aortic smooth muscle cells were cultured in vitro under conditions which allow phenotypic modulation, i.e. favouring either the ‘contractile’ or the ‘synthetic’ vascular smooth muscle phenotype ((2007) Annu Rev Pathol 2007; 2:369-99). A time course of continuous culture for 7 days before the cells were harvested and samples were analysed for protein levels of Nkx2-5, collagen type I and the smooth muscle cells marker proteins (α-SMA, smooth muscle myosin heavy chain (smMHC or MYH11), smoothelin). Aortic smooth muscle cells cultured under ‘contractile’conditions, express little or no Nkx2-5 protein and low levels of collagen type I. In contrast, the levels of the contractile proteins were high. Over time and as the cell phenotype becomes more ‘synthetic’, Nkx2-5 protein expression becomes apparent and collagen type I expression increases in parallel whilst the levels of contractile proteins were down-regulated (FIG. 13A). Changes in protein are mirrored by increased mRNA levels (data not shown). Inhibition of Nkx2-5 expression by siRNA in ‘synthetic’ aortic smooth muscle cells not only down-regulate collagen type I protein levels but other proteins associated with the extracellular matrix that are known to be upregulated as part of the fibrogenic response (CTGF, Fibronectin). At the same time Nkx2-5 knockdown results in the up-regulation of the expression of proteins associated with the normal differentiated, contractile state of the smooth muscle cell (α-SMA, sm-MHC, smoothelin) (FIG. 13B). Indeed, four colour immunofluorescent staining of Human aortic smooth muscle cells reveals that inhibition of Nkx2-5 using siRNA results in downregulation of procollagen type I and up-regulation of αSMA fibres and an obvious change in smooth muscle cell phenotype (FIG. 13C). These data suggest that Nkx2-5 is directly involved in the de-differentiation of vascular smooth muscle cells from the differentiated contractile phenotype to the de-differentiated synthetic phenotype associated with vascular pathology, remodelling and scarring.

Three-colour immunofluorescent staining was carried out of human smooth muscle with Nkx2-5 specific, pro collagen type I specific primary antibodies. Secondary fluorescently labelled antibodies (red and green) were used to visualise Nkx2-5 and procollagen I respectively and DAPI (blue) to visualise the cell nuclei (FIG. 14). The procollagen antibody detects the intracellular procollagen type I pro-peptide but does not detect mature extracellularly secreted collagen type I. Neither Nkx2-5 or procollagen type I are expressed in contractile smooth muscle cells in vitro and only the DAPI stained nuclei can be detected (FIG. 14A). If the cells are grown under conditions favouring the synthetic phenotype for 2 days (FIG. 14B) expression of Nkx2-5 can be observed in the cytoplasm and in the nucleus. At the same time, low levels of procollagen type I can be detected in the cytoplasm (FIG. 14B). If the cells are left in culture under synthetic conditions for 5 days (FIG. 14C), Nkx2-5 expression is mostly nuclear and procollagen type I levels are much higher. Nkx2-5 antibody specificity was controlled by using the Nkx2-5 antibody previously mixed with a peptide to which it was raised provided by the manufacturers (FIG. 14D). Nkx2-5 staining which had previously been detected after 5 days of culture under synthetic conditions was abolished using the pre-adsorbed Nkx2-5 antibody. Procollagen type I detection was unaffected (FIG. 14D).

### Example 15

### Function of Nkx2-5 in Vascular Smooth Muscle Cells

Expression of Nkx2-5 in vascular smooth muscle cells has an anti-contractile effect. Human aortic smooth muscle cells were cultured under conditions favouring either the ‘contractile’ or the ‘synthetic’ phenotype and treated with either control siRNAs or siNkx2-5. They were then seeded in a collagen type I matrix and a loaded in a cell Tensioning-Culture Force Monitor (t-CFM) (REF). The t-CFM is an apparatus which applies physiological loads to 3D matrices and measures a) the initial force generated by the cells within the matrix due to integrin attachment and cytoskeletal formation and b) the maintenance of force within the cell termed Tensional Homeostasis, i.e. tension generated within the cell by microfilaments balanced by microtubules and the surrounding collagen matrix. The effect of knocking down Nkx2-5 in contractile cells was not significant (FIG. 15A). This is to be expected because there is very low Nkx2-5 expression in these cells. ‘Synthetic’ cells by definition are less contractile than ‘contractile’ cells (FIG. 15A). The effect of inhibiting Nkx2-5 is greater compared to the control, resulting in a considerable increase in the force exerted and therefore the contractility of the cells (FIG. 15A).

Another function of Nkx2-5 in vascular smooth muscle cells is to promote migration. Human aortic smooth muscle cells were again cultured under conditions favouring either the ‘contractile’ or the ‘synthetic’ phenotype and treated with either control siRNAs or siNkx2-5. A scratch migration study was then carried out in the presence/absence of the proliferation inhibitor Mitomycin c. Not surprisingly, there was not a significant effect in the rate of cell migration into the scratch between siControl and siNkx2-5 treated cells. This is expected as Nkx2-5 expression levels are very low in these cells (FIG. 15B). Indeed, there was no significant effect of Nkx2-5 inhibition ‘synthetic’ in migration assays carried out in the absence of mitomycin suggesting that Nkx2-5 does not play a role in the proliferation of these cells (FIG. 15C). However in the presence of mitomycin, Nkx2-5 inhibition results in a highly significant decrease of the rate of cell migration into the scratch (FIG. 15C). This suggests a pro-migratory role for Nkx2-5 in human vascular smooth muscle cells.

### Example 16

### Effect of CK2 Antagonists on Nkx2-5 Nuclear Localisation

The potency of various casein Kinase II (CK2) antagonists to inhibit nuclear localisation of NKX2-5 was assessed in primary disease relevant human pulmonary arterial smooth muscle cells (hPASMCs). HPASMCs were incubated in the presence or absence of CK2 antagonists at IC80 for 24 hrs and the effects of nuclear translocation of NKX2-5 determined by immunofluorescence using a Nkx2-5 specific antibody followed by a fluorescent secondary antibody (Alexa 594) and DAPI (nuclear staining). Antagonists tested were CK2 Inhibitors IV to VIII (CK2 Inv IV to VIII) and CX-4945. The number of Nkx2-5 positive nuclei were expressed as a percentage of the total number of cells (DAPI stained nuclei) in 4 separate fields of view per treatment.*** P<0.001 by unpaired two-tailed Student's t test. Error bars, s.e.m. The data are shown in FIG. 16, and in Table 2. As can be seen, all CK2 compounds inhibited Nkx2-5 nuclear localisation, with CX-4945 exerting the most potent repression of Nkx2-5 nuclear localisation.

TABLE 2
IC50 and IC80 values for CK2 antagonists

| Compound name | Cat No | IC50 (nM) | IC80 (nM) |
|---|---|---|---|
| CK2 Inhibitor VIII | 218860 (Calbiochem) | 46 | 73.6 |
| (CK2 Inv VIII) | | | |
| CK2 Inhibitor VI | 218718 (Calbiochem) | 500 | 800 |
| (CK2 Inv VI) | | | |
| CK2 Inhibitor V | 218717 (Calbiochem) | 110 | 176 |
| (CK2 Inv V) | | | |
| CK2 Inhibitor III | 218710 (Calbiochem) | 110 | 176 |
| (CK2 Inv III) | | | |
| CK2 Inhibitor IV | 218713 (Calbiochem) | 9 | 14.4 |
| (CK2 Inv IV) | | | |
| CX-4945 | | 1 | 1.6 |

### Example 17

### Effect of CX-4945 Antagonism on Right Ventricular Systolic Pressure (RVSP) and Systemic Pressures in a Pre-Clinical Model of PAH

The effect of the CK2 antagonist CX-4945 was assessed in the hypoxia sugen pre-clinical model of pulmonary hypertension as described in Am J Respir Crit Care Med. 2011. Female C57/B16 were maintained in normoxia or hypoxia (10% 02) in a ventilated chamber for 20 days and dosed with CX-4945 (80 mg/kg) or vehicle daily. Prophylactic (pro) administration was performed for the full 20 days and therapeutic (ther) administration of CX-4945 (80 mg/kg) was commenced 8 days post experimental initiation. The data are shown in FIG. 17. Right ventricular systolic pressure (RVSP, left panel) and mean arterial blood pressure (MABP, right panel) was determined and plotted. Data are mean±SEM of a minimum of 5 animals per group. Statistical differences are indicated. As can be seen in FIG. 17, administration of CX-4945 significantly inhibited RVSP increases in the hypoxia sugen pre-clinical model of pulmonary hypertension when administered prophylactically (pro) or therapeutically (ther) when compared to hypoxia sugen controls (left panel). No significant effects by CX-4945 on systemic pressure was observed (right panel).

### Example 18

### Effect of Nkx2-5 Deletion on Vascular Remodelling in the Femoral Artery Ligation Model

The inventors wanted to test whether deletion of Nkx2-5 alters remodelling of vessels following injury of the systemic vasculature. They utilised the femoral artery ligation mouse model which result in neointimal hyperplasia. Reduced blood flow by complete ligation of the femoral arteries induced rapid proliferation of medial VSMC, leading to extensive neointima lesion formation. They ligated femoral arteries of Nkx2-5^{flox} Cre⁺ or Nkx2-5^{flox} Cre⁻ mice given tamoxifen or Nkx2-5^{flox} Cre⁺ on a normal diet. The data are shown in FIG. 18. After 4 weeks of ligation, they observed high levels of Nkx2-5 expression in the media and neointima of control mice (B) and the luminal area was reduced through an increase in neointima formation in control in control mice (A). In media of Nkx2-5^{flox} Cre⁺ in the presence of tamoxifen mice, Nkx2-5 was deleted, the luminal area was significantly greater (p<0.00012) and the neointimal lesion was significantly smaller (p<0.000004). There was no significant alteration in morphology between the control groups.

### SUMMARY

The inventors have summarised their findings as follows:—

1) Nkx2-5 is not expressed by normal vessels. It is expressed in remodelling vessels and during vessel pathology.
2) Nkx2-5 has been observed in the human atherosclerotic lesions in the aorta, the coronary, the carotid and the femoral arteries. It is also expressed in the pulmonary vasculature of patients with pulmonary arterial hypertension.
3) In the chronic hypoxia mouse model of pulmonary arterial hypertension (PAH), conditional knockout of Nkx2-5 in collagen-producing cells reverses the features of PAH associated with this model, resulting in a significant decrease of pulmonary vessel muscularisation, RSVP and right ventricular hypertrophy. In addition the pulmonary vessels are more compliant.
4) In the apoE null mouse of atherosclerosis, Nkx2-5 is produced by collagen-producing smooth muscle cells and not by macrophages or known progenitor cell populations. It is predominantly expressed in the fibrous cap and shoulders of the lesions although it is also expressed in the media and within the lesion itself.
5) In the carotid ligation model of vascular remodelling, conditional knockout of Nkx2-5 in collagen-producing cells results in a highly significant decrease in neointima formation.
6) Nkx2-5 expression in vascular smooth muscle cells promotes phenotypic modulation and the de-differentiation from a contractile to a synthetic phenotype, promoted proliferation, extracellular matrix protein expression and inhibits contractile protein expression, and has an anti-contractile and pro-migratory effect.
7) Nkx2-5 is regulated by phosphorylation by casein kinase II (CK II). Nkx2-5 phosphorylation results in a decrease of Nkx2-5 nuclear translocation and DNA binding ability. CK II inhibitor CX-4945, inhibits Nkx2-5 nuclear translocation at a concentration range 1-10 nM in vascular smooth muscle cells. It blocks phenotypic modulation from a normal ‘contractile’ to the ‘synthetic’ vascular smooth muscle cells phenotype associated with vascular remodelling and pathology and inhibits vascular smooth muscle cell proliferation and migration.
8) The data supports inhibition of Nkx2-5 activity by CK II inhibitors (e.g. CX-4945) will have important therapeutic implications in the treatment of diseases with vascular remodelling components including pulmonary hypertension/pulmonary arterial hypertension, atherosclerosis, coronary artery disease (CAD), peripheral arterial disease (PAD), chronic limb ischemia and stroke.
9) The data supports that NKX2-5 is essential in vascular remodelling in the limb (hind limb ischemia model) and thus supports the claim that NKX2-5 is mediator of vascular remodelling diseases.
10) CX4945 represents the most potent CKII antagonist (based on in vitro assessment).
11) CKII antagonism using CX4945 can inhibit and reverse the development of pulmonary hypertension in a pre-clinical model.

1-20. (canceled)

21. A method of treating, ameliorating or preventing a disease characterised by inappropriate vascular remodelling in a subject, the method comprising administering, to a subject in need of such treatment, a therapeutically effective amount of an inhibitor of Nkx2.5 activity.

22. The method according to claim 21, wherein the inhibitor:
(a) reduces interaction between Nkx2-5 and nucleic acid and/or other transcription factors;
(b) competes with endogenous Nkx2-5 for nucleic acid binding and/or other transcription factor binding;
(c) binds to Nkx2-5 to reduce its biological activity;
(d) decreases the expression of Nkx2-5; or
(e) inhibits Nkx2-5 translocation to the nucleus.

23. The method according to claim 21, wherein the inhibitor prevents or reduces expression of Nkx2-5, and wherein Nkx2-5 comprises an amino acid sequence substantially as set out in any one of SEQ ID No: 2, 3, or 4, or a functional variant or fragment thereof.

24. The method according to claim 23, wherein the inhibitor is a gene-silencing molecule.

25. The method according to claim 24, wherein the gene-silencing molecule is an siRNA.

26. The method according to claim 24, wherein the gene-silencing molecule is selected from:
(SEQ ID No. 5)
5′ - CCTCAATCCCTACGGTTAT - 3′;
(SEQ ID No. 6)
5′ - CCAACAACAACTTCGTGAA - 3′;
(SEQ ID No. 7)
5′ - GCTACAAGTGCAAGCGGCA - 3′;
and
(SEQ ID No. 8)
5′ - CCGGGATTCCGCAGAGCAA - 3′.

27. The method according to claim 24, wherein the gene silencing molecule is an miRNA which is selected from: miR-125b, miR-145, miR-143, miR-367, miR-384, miR-363, miR-32, miR-25, and miR-92a and b.

28. The method according to claim 21, wherein the inhibitor is capable of inhibiting casein kinase II (CK II) activity.

29. The method according to claim 28, wherein the CK II inhibitor is selected from: CX-4945 [CAS number 1009820-21-6]; CX-8184; Casein Kinase II Inhibitor III, (TBCA) [CAS 934358-00-6]; CKII inhibitor IV (IQA) [CAS 391670-48-7]; Casein Kinase II Inhibitor V, (Quinalizarin) [CAS 81-61-8]; Casein Kinase II Inhibitor VI, (TMCB) [CAS 905105-89-7], Casein Kinase II Inhibitor VII, and Casein Kinase II Inhibitor VIII.

30. The method according to claim 28, wherein the CK II inhibitor comprises CX-4945.

31. The method according to claim 21, wherein the disease characterised by inappropriate vascular remodelling and deposition of vascular extracellular matrix, which is treated, is: pulmonary hypertension (PH), pulmonary arterial hypertension (PAH) including all types of PAH associated with connective tissue diseases or HIV, atherosclerosis, coronary artery disease (CAD), peripheral arterial disease (PAD), chronic limb ischemia or stroke, renal artery disease, metabolic syndrome and diabetes, rheumatological diseases (e.g. systemic lupus erethematosus, systemic sclerosis, rheumatoid arthritis, vasculis), fibromuscular dysplasia or aneurisms.

32. The method according to claim 21, for treating pulmonary arterial hypertension (PAH), atherosclerosis, chronic limb ischemia or stroke.

33. An inappropriate vascular remodelling treatment composition, comprising an inhibitor of Nkx2.5 activity as defined in claim 21, and a pharmaceutically acceptable vehicle.

34. A process for making the composition according to claim 33, the process comprising contacting a therapeutically effective amount of an inhibitor of Nkx2.5 activity and a pharmaceutically acceptable vehicle.

35. An assay for screening a test compound to test whether or not the compound has efficacy for treating or preventing a disease characterised by inappropriate vascular remodelling, the assay comprising:
(a) exposing a biological system to a test compound;
(b) detecting the activity or expression of Nkx2-5 in the biological system; and
(c) comparing the activity or expression of Nkx2-5 in the biological system treated with the test compound relative to the activity or expression of Nkx2-5 found in a control biological system that was not treated with the test compound,
wherein a decreased level of activity or expression of Nkx2-5 in the presence of the test compound relative to that detected in the control biological system is an indication of the ability of the test compound to treat or prevent a disease characterised by inappropriate vascular remodelling.

36. An assay for screening a test compound to test whether or not the compound causes a disease characterised by inappropriate vascular remodelling, the assay comprising:
(a) exposing a biological system to a test compound;
(b) detecting the activity or expression of Nkx2-5 in the biological system; and
(c) comparing the activity or expression of Nkx2-5 in the biological system treated with the test compound relative to the activity or expression of Nkx2-5 found in a control biological system that was not treated with the test compound,
wherein an increased level of activity or expression of Nkx2-5 in the presence of the test compound relative to that detected in the control biological system is an indication that the test compound causes a disease characterised by inappropriate vascular remodelling.


SEQUENCE LISTING (8 sequences)

SEQ ID NO: 1 - DNA, 3209 bp, Homo sapiens
gctcctgtcatcgaggcccctggcccaatggcaggctgagtccccctcctctggcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaaggtgaggaggaaacacaggcccccttctcccctcctgggtcgctttcgtccccaagaaactcagggccaggaggaggagacacgcgcccttgggccgagggctgggctgcggcggggggttcagaatgtaagatgcctggtgttgtcgccaggctcccgcgccccgcgtccaatcggaggttcagaggaaatgccggattgaaaggatcagaagcaagagaccaaaaaacgtttccccccggcctaacaaagccccgggcggcttcggctctgctcctgggtctggtaggaagttgagaaatcggtttatggtagacagaacagagagacaagcagataatctctgtttttaaatctcctttggatttacgaatctttttaaagatctgatgagaaccgctaaacagaaattgaaatgttgctcaccagacagcttttgcgtacaatcggaggagggtcctggaccttctttctgcagcccacccacgacccgggtttctggtgcctttctttctttgcgccaggaaagtggagtctgggatcgagggccttgattttaaaatgggatactgcggaccctcaggaatctgacttcactttattttttcagcacaacttgccggcgcggccagggcggagaggttccctcgtggaaaagttaggaaatgctgcgctaccgcgggcacaagggagtggacgagatgagtgcgggatcatcccgcaggccatcccaggatcggggagggaggccggccccgctgcagaaaggggccttctgggagaccccccagcccaaggcaggagcccgggcgattcccgggaggccgcaggcgctgggcgaagcgctgggcgaagggccgctgccagccgggagagaattcataggtttgttgaggagcagaggcctgggaacaaattcgggcgggcacggcggctagaactgatcgctaccaattcgaggaagccagcaaggcaggttccgaggccgcctgcccacccgcagcttcttggacactgcgcaaaccctgctgcggccaggctggagcctccgatcaccaaaccaacactccctggccttctgtttcttgattccttaattttgagataagaccgtccctagcagtgaggcctcggcctctgttcatttaacttctcaaaccaaactagccctaattcagttcaccccagagcatcacctggttttatttttatttttttatttttttatttattttttttttttttgcagcctgaaattttaagtcaccgtctgtctccctcaccagggtgtgaactgccccgagggcagagacctcccgttttgttctccagcgccttgagccagcctgactttctacaaatgctgagtgagacgtgtcggtggctcccagtgcacttggcagagtgagccgcagccagctgggcgctccaggcaggacacagtggcctccacgaggatcccttaccattactgtgcggccgcgctccgtaggtcaagccgctcttaccaagcgtctctctgcctctctgttccccctcagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatccccttcccattaaagagagtgcgttgaccccg

SEQ ID NO: 2 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 3 - protein, 112 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKA

SEQ ID NO: 4 - protein, 151 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKGCELPRGQRPPVLFSSALSQPDFLQMLSETCRWLPVHLAE

SEQ ID NO: 5 - DNA, 19 bp, Homo sapiens
cctcaatccctacggttat

SEQ ID NO: 6 - DNA, 19 bp, Homo sapiens
ccaacaacaacttcgtgaa

SEQ ID NO: 7 - DNA, 19 bp, Homo sapiens
gctacaagtgcaagcggca

SEQ ID NO: 8 - DNA, 19 bp, Homo sapiens
ccgggattccgcagagcaa